SEQUENCE LISTING (81 sequences)

SEQ ID NO: 1 - protein, 98 aa, Artificial Sequence
  AC1
MDAEDLLLKLNLAATVGTAPPKGKNLGQAYPASSDKESEVGRYSHSPHQGSSACMVARRRRRRAHRDGMACPSTRSNNGIAIPVPTAALLIILGGDDI

SEQ ID NO: 2 - protein, 325 aa, Artificial Sequence
  cpDKK3b fusion protein
MRGSLKHHHHHHLKGMASMTGGQQMKLGDIYARAAARQARADIGGSTMEAEEAAAKASSEVNLANLPPSYHNETNTDTKVGNNTIHVHREIHKITNNQTGMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALREPAAAAAALLGGEEI

SEQ ID NO: 3 - protein, 67 aa, Artificial Sequence
  DKK1 Residues 74-141
QTIDNYQPYPCAEDEECGTDEYCASPTRGGDAGVQICLACRKRRKRCMRHAMCCPGNYCKNGICVSS

SEQ ID NO: 4 - protein, 53 aa, Artificial Sequence
  DKK2 Residues 78-130
CSSDKECEVGRYCHSPHQGSSACMVCRRKKKRCHRDGMCCPSTRCNNGICIPV

SEQ ID NO: 5 - protein, 53 aa, Artificial Sequence
  DKK3b Residues 74-126
HECIIDEDCGPSMYCQFASFQYTCQPCRGQRMLCTRDSECCGDQLCVWGHCTK

SEQ ID NO: 6 - protein, 61 aa, Artificial Sequence
  DKK4 Residues 31-91
DLHGARKGSQCLSDTDCNTRKFCLQPRDEKPFCATCRGLRRRCQRDAMCCPGTLCVNDVCT

SEQ ID NO: 7 - protein, 53 aa, Artificial Sequence
  Cysteine deficient DKK3b OR Modified DKK3b
HEAIIDEDSGPSMYSQFASFQYTCQPARRRRRRATRDSESCGDQLAVWGHSTK

SEQ ID NO: 8 - protein, 67 aa, Artificial Sequence
  Cysteine deficient DKK1 OR Modified DKK1
QTIDNYQPYPAAEDEESGTDEYSASPTRGGDAGVQICLAARRRRRRAMRHAMSCPGNYAKNGIAVSS

SEQ ID NO: 9 - protein, 7 aa, Artificial Sequence
  C. elegans SDC3
FKKFRKF

SEQ ID NO: 10 - protein, 19 aa, Artificial Sequence
  CADY-K
GLWRALWRLLRSLWRLLWK

SEQ ID NO: 11 - protein, 23 aa, Artificial Sequence
  EB1 CPP
LIRLWSHLIHIWFQNRRLKWKKK

SEQ ID NO: 12 - protein, 27 aa, Artificial Sequence
  FBP CPP
GALFLGWLGAAGSTMGAWSQPKKKRKV

SEQ ID NO: 13 - protein, 16 aa, Artificial Sequence
  FGF4 CPP
AAVALLPAVLLALLAP

SEQ ID NO: 14 - protein, 8 aa, Artificial Sequence
  HATF3
ERKKRRRE

SEQ ID NO: 15 - protein, 22 aa, Artificial Sequence
  hCT CPP
LGTYTQDFNKTFPQTAIGVGAP

SEQ ID NO: 16 - protein, 18 aa, Artificial Sequence
  MAP CPP
KLALKLALKALKAALKLA

SEQ ID NO: 17 - protein, 27 aa, Artificial Sequence
  MPG CPP
GLAFLGFLGAAGSTMGAWSQPKKKRKV

SEQ ID NO: 18 - protein, 10 aa, Artificial Sequence
  NF-kB
VQRKRQKLMP

SEQ ID NO: 19 - protein, 8 aa, Artificial Sequence
  OCT-6
GRKRKKRT

SEQ ID NO: 20 - protein, 16 aa, Artificial Sequence
  Penetratin CPP
RQIKIWFQNRRMKWKK

SEQ ID NO: 21 - protein, 16 aa, Artificial Sequence
  Penetratin CPP variant 1
RQLKLWFQNRRMKWKK

SEQ ID NO: 22 - protein, 16 aa, Artificial Sequence
  Penetratin CPP variant 2
REIKIWFQNRRMKWKK

SEQ ID NO: 23 - protein, 20 aa, Artificial Sequence
  Pep-1 CPP
KETWWETWWTEWSQPKKRKV

SEQ ID NO: 24 - protein, 16 aa, Artificial Sequence
  pIsl CPP
PVIRVWFQNKRCKDKK

SEQ ID NO: 25 - protein, 10 aa, Artificial Sequence
  Poly-Arg CPP
  MISC_FEATURE (5)..(10): Xaa at positions 5-10 are Arg and up to six of them may be absent
RRRRXXXXXX

SEQ ID NO: 26 - protein, 16 aa, Artificial Sequence
  pVEC CPP
LLIILRRRIRKQAHAH

SEQ ID NO: 27 - protein, 16 aa, Artificial Sequence
  RL-16 CPP
RRLRRLLRRLLRRLRR

SEQ ID NO: 28 - protein, 12 aa, Artificial Sequence
  RVG CPP
RVGRRRRRRRRR

SEQ ID NO: 29 - protein, 9 aa, Artificial Sequence
  R6W3 CPP
RRWWRRWRR

SEQ ID NO: 30 - protein, 27 aa, Artificial Sequence
  SBP CPP
MGLGLHLLVLAAALQGAWSQPKKKRKV

SEQ ID NO: 31 - protein, 7 aa, Artificial Sequence
  SV40
PKKKRKV

SEQ ID NO: 32 - protein, 17 aa, Artificial Sequence
  SynB1 CPP
RGGRLSYSRRFSTSTGR

SEQ ID NO: 33 - protein, 10 aa, Artificial Sequence
  SynB3 CPP
RRLSYSRRRF

SEQ ID NO: 34 - protein, 17 aa, Artificial Sequence
  SynB5 CPP
RGGRLAYLRRRWAVLGR

SEQ ID NO: 35 - protein, 11 aa, Artificial Sequence
  Tat47-57 CPP
YGRKKRRQRRR

SEQ ID NO: 36 - protein, 10 aa, Artificial Sequence
  Tat47-56 CPP
YGRKKRRQRR

SEQ ID NO: 37 - protein, 9 aa, Artificial Sequence
  Tat48-56 CPP
GRKKRRQRR

SEQ ID NO: 38 - protein, 13 aa, Artificial Sequence
  Tat48-60 CPP
GRKKRRQRRRPPQ

SEQ ID NO: 39 - protein, 11 aa, Artificial Sequence
  TCF1-a
GKKKKRKREKL

SEQ ID NO: 40 - protein, 8 aa, Artificial Sequence
  TFIIE-b
SKKKKTKV

SEQ ID NO: 41 - protein, 27 aa, Artificial Sequence
  TP CPP
GWTLNSAGYLLGKINLKALAALAKKIL

SEQ ID NO: 42 - protein, 21 aa, Artificial Sequence
  TP10 CPP
AGYLLGKINLKALAALAKKIL

SEQ ID NO: 43 - protein, 12 aa, Artificial Sequence
  TP2 CPP
PLIYLRLLRGQF

SEQ ID NO: 44 - protein, 34 aa, Artificial Sequence
  VP22 CPP
DAATATRGRSAASRPTQRPRAPARSASRPRRPVQ

SEQ ID NO: 45 - protein, 63 aa, Artificial Sequence
  Cysteine deficient DKK2 OR Modified DKK2
KGKNLGQAYPASSDKESEVGRYSHSPHQGSSACMVARRRRRRAHRDGMACPSTRSNNGIAIPV

SEQ ID NO: 46 - protein, 61 aa, Artificial Sequence
  Cysteine deficient DKK4 OR Modified DKK4
DLHGARKGSQALSDTDSNTRKFSLQPRDEKPFCATARRRRRRAQRDAMSCPGTLSVNDVAT

SEQ ID NO: 47 - protein, 279 aa, Homo sapiens
MEAEEAAAKASSEVNLANLPPSYHNETNTDTKVGNNTIHVHREIHKITNNQTGQMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALREPAAAAAALLGGEEI

SEQ ID NO: 48 - protein, 21 aa, Artificial Sequence
  N-terminal domain
MEADELLLKLNLAATVGFAPP

SEQ ID NO: 49 - protein, 13 aa, Artificial Sequence
  C-terminal domain
TSQLLIILGGDDI

SEQ ID NO: 50 - protein, 137 aa, Artificial Sequence
  DKK3b peptide mimetic including SRP and poly-His tag
MTRLTVLALLAGLLASSRAGSGRGHHHHHHVGTGSNSPGMDAEDLLLKLNLAATVGTAPPKGKNLGQAYPASSDKESEVGRYSHSPHQGSSACMVARRRRRRAHRDGMACPSTRSNNGIAIPVPTAALLIILGGDDI

SEQ ID NO: 51 - protein, 14 aa, Artificial Sequence
  Variant cell penetrating domain
WLRRIKAWLRRIKA

SEQ ID NO: 52 - protein, 11 aa, Artificial Sequence
  Variant cell penetrating domain
YGRKKRRQRRR

SEQ ID NO: 53 - protein, 11 aa, Artificial Sequence
  Variant cell penetrating domain
YARAAARQARA

SEQ ID NO: 54 - protein, 17 aa, Artificial Sequence
  DKK1 cell penetrating domain
CLACRKRRKRCMRHMCC

SEQ ID NO: 55 - protein, 18 aa, Artificial Sequence
  DKK2 cell penetrating domain
CMVCRRKKKRCHRDGMCC

SEQ ID NO: 56 - protein, 18 aa, Artificial Sequence
  DKK3B cell penetrating domain
CQPCRGQRMLCTRDSECC

SEQ ID NO: 57 - protein, 18 aa, Artificial Sequence
  DKK4 cell penetrating domain
CATCRGLRRRCQRDAMCC

SEQ ID NO: 58 - protein, 326 aa, Artificial Sequence
  cpDKK3b fusion protein
MRGSLKHHHHHHLKGMASMTGGQQMKLGDIYARAAARQARADIGGSTMEAEEAAAKASSEVNLANLPPSYHNETNTDTKVGNNTIHVHREIHKITNNQTGQMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALREPAAAAAALLGGEEI

SEQ ID NO: 59 - protein, 21 aa, Artificial Sequence
  N-terminal domain
MDAEDLLLKLNLAATVGTAPP

SEQ ID NO: 60 - protein, 13 aa, Artificial Sequence
  N-terminal domain
TAALLIILGGDDI

SEQ ID NO: 61 - protein, 20 aa, Artificial Sequence
  N-terminal domain
DAEDLLLKLNLAATVGTAPP

SEQ ID NO: 62 - protein, 21 aa, Artificial Sequence
  N-terminal domain
  MISC_FEATURE (1)..(1): nonpolar amino acid other than proline; or is serine or threonine; in certain aspects, is threonine, serine or a nonpolar amino acid other than proline and methionine; is alanine or isoleucine
XDAEDLLLKLNLAATVGTAPP

SEQ ID NO: 63 - protein, 21 aa, Artificial Sequence
  N-terminal domain
ADAEDLLLKLNLAATVGTAPP

SEQ ID NO: 64 - protein, 21 aa, Artificial Sequence
  N-terminal domain
IDAEDLLLKLNLAATVGTAPP

SEQ ID NO: 65 - protein, 97 aa, Artificial Sequence
  DKK3b peptide mimetic
DAEDLLLKLNLAATVGTAPPKGKNLGQAYPASSDKESEVGRYSHSPHQGSSACMVARRRRRRAHRDGMACPSTRSNNGIAIPVPTAALLIILGGDDI

SEQ ID NO: 66 - protein, 98 aa, Artificial Sequence
  DKK3b peptide mimetic
  MISC_FEATURE (1)..(1): nonpolar amino acid other than proline; or is serine or threonine; in certain aspects, is threonine, serine or a nonpolar amino acid other than proline and methionine
XDAEDLLLKLNLAATVGTAPPKGKNLGQAYPASSDKESEVGRYSHSPHQGSSACMVARRRRRRAHRDGMACPSTRSNNGIAIPVPTAALLIILGGDDI

SEQ ID NO: 67 - protein, 98 aa, Artificial Sequence
  DKK3b peptide mimetic
ADAEDLLLKLNLAATVGTAPPKGKNLGQAYPASSDKESEVGRYSHSPHQGSSACMVARRRRRRAHRDGMACPSTRSNNGIAIPVPTAALLIILGGDDI

SEQ ID NO: 68 - protein, 98 aa, Artificial Sequence
  DKK3b peptide mimetic
IDAEDLLLKLNLAATVGTAPPKGKNLGQAYPASSDKESEVGRYSHSPHQGSSACMVARRRRRRAHRDGMACPSTRSNNGIAIPVPTAALLIILGGDDI

SEQ ID NO: 69 - protein, 53 aa, Artificial Sequence
  DKK2 variant
ASSDKESEVGRYSHSPHQGSSACMVARRRRRRAHRDGMACPSTRSNNGIAIPV

SEQ ID NO: 70 - protein, 98 aa, Artificial Sequence
  DKK3b peptide mimetic
  MISC_FEATURE (1)..(1): nonpolar amino acid other than proline; or is serine or threonine; in certain aspects, is threonine, serine or a nonpolar amino acid other than proline and methionine
  MISC_FEATURE (54)..(54): serine or alanine
  MISC_FEATURE (71)..(71): serine or alanine
XDAEDLLLKLNLAATVGTAPPKGKNLGQAYPASSDKESEVGRYSHSPHQGSSAXMVARRRRRRAHRDGMAXPSTRSNNGIAIPVPTAALLIILGGDDI

SEQ ID NO: 71 - protein, 98 aa, Artificial Sequence
  DKK3b peptide mimetic
  MISC_FEATURE (54)..(54): serine or alanine
  MISC_FEATURE (71)..(71): serine or alanine
ADAEDLLLKLNLAATVGTAPPKGKNLGQAYPASSDKESEVGRYSHSPHQGSSAXMVARRRRRRAHRDGMAXPSTRSNNGIAIPVPTAALLIILGGDDI

SEQ ID NO: 72 - protein, 98 aa, Artificial Sequence
  DKK3b peptide mimetic
  MISC_FEATURE (54)..(54): serine or alanine
  MISC_FEATURE (71)..(71): serine or alanine
IDAEDLLLKLNLAATVGTAPPKGKNLGQAYPASSDKESEVGRYSHSPHQGSSAXMVARRRRRRAHRDGMAXPSTRSNNGIAIPVPTAALLIILGGDDI

SEQ ID NO: 73 - protein, 53 aa, Artificial Sequence
  DKK2 variant
CSSDKECEVGRYCHSPHQGSSACMVCRRRRRRCHRDGMCCPSTRCNNGICIPV

SEQ ID NO: 74 - protein, 53 aa, Artificial Sequence
  N-1 domain variant
  MISC_FEATURE (23)..(23): alanine or serine
  MISC_FEATURE (40)..(40): alanine or serine
ASSDKESEVGRYSHSPHQGSSAXMVARRRRRRAHRDGMAXPSTRSNNGIAIPV

SEQ ID NO: 75 - protein, 98 aa, Artificial Sequence
  DKK3b peptide mimetic
IDAEDLLLKLNLAATVGTAPPKGKNLGQAYPASSDKESEVGRYSHSPHQGSSASMVARRRRRRAHRDGMAAPSTRSNNGIAIPVPTAALLIILGGDDI

SEQ ID NO: 76 - protein, 98 aa, Artificial Sequence
  DKK3b peptide mimetic
IDAEDLLLKLNLAATVGTAPPKGKNLGQAYPASSDKESEVGRYSHSPHQGSSAAMVARRRRRRAHRDGMASPSTRSNNGIAIPVPTAALLIILGGDDI

SEQ ID NO: 77 - protein, 98 aa, Artificial Sequence
  DKK3b peptide mimetic
ADAEDLLLKLNLAATVGTAPPKGKNLGQAYPASSDKESEVGRYSHSPHQGSSASMVARRRRRRAHRDGMAAPSTRSNNGIAIPVPTAALLIILGGDDI

SEQ ID NO: 78 - protein, 98 aa, Artificial Sequence
  DKK3b peptide mimetic
ADAEDLLLKLNLAATVGTAPPKGKNLGQAYPASSDKESEVGRYSHSPHQGSSAAMVARRRRRRAHRDGMASPSTRSNNGIAIPVPTAALLIILGGDDI

SEQ ID NO: 79 - protein, 98 aa, Artificial Sequence
  DKK3b peptide mimetic
MDAEDLLLKLNLAATVGTAPPKGKNLGQAYPASSDKESEVGRYSHSPHQGSSASMVARRRRRRAHRDGMAAPSTRSNNGIAIPVPTAALLIILGGDDI

SEQ ID NO: 80 - protein, 98 aa, Artificial Sequence
  DKK3b peptide mimetic
MDAEDLLLKLNLAATVGTAPPKGKNLGQAYPASSDKESEVGRYSHSPHQGSSAAMVARRRRRRAHRDGMASPSTRSNNGIAIPVPTAALLIILGGDDI

SEQ ID NO: 81 - protein, 12 aa, Artificial Sequence
  Poly-Arg CPP
  MISC_FEATURE (7)..(12): Xaa at positions 7-12 are Arg and up to five of them may be absent
RRRRRRXXXXXX

(19) United States
(12) Patent Application Publication
(10) Pub. No.: US 2022/0332793 A1
(43) Pub. Date: Oct. 20, 2022

(54) PEPTIDE MIMETICS OF DKK3b AND METHODS OF USE

(71) Applicant: Acworth Pharmaceuticals, Inc., Cambridge, MA (US)

(72) Inventor: Jack L. Leonard, Shrewsbury, MA (US)

(21) Appl. No.: 17/725,608

(22) Filed: Apr. 21, 2022

Related U.S. Application Data
(63) Continuation of application No. PCT/US20/57841, filed on Oct. 29, 2020.
(60) Provisional application No. 62/927,328, filed on Oct. 29, 2019.

(51) Int. Cl. C07K 14/71

(52) U.S. Cl. CPC C07K 14/71; C07K 2319/033

(57) ABSTRACT

The invention provides highly potent and stable peptide mimetics of human DKK3b having numerous improved properties. The peptide mimetics of the invention are useful as therapeutics in the treatment of various diseases wherein inhibiting nuclear translocation of β-catenin is therapeutic, including, but not limited to, cancer/proliferative, metabolic, osteoporosis, neurological, immunological, endocrinologic, cardiovascular, hematologic, and inflammatory diseases.

### RELATED APPLICATIONS

This application is a continuation of International Application No. PCT/US20/057841, which designated the United States and was filed on Oct. 29, 2020, published in English, which claims the benefit of U.S. Provisional Application No. 62/927,328 filed Oct. 29, 2019. The entire contents of the above applications are incorporated by reference herein.

### BACKGROUND OF THE INVENTION

Wnt/β-catenin signaling is a vital cellular regulatory pathway that impacts embryogenesis, organogenesis, and maintains tissue and organ homeostasis. It is also integral in several physiological events such as differentiation, proliferation, survival, oxidative stress, morphogenesis, and others.

However, aberrant activation of this pathway is responsible for multiple pathological conditions. β-catenin stabilization and subsequent translocation to the nucleus, often in response to Wnt activation of the Frizzled receptor, is a key step that is dysregulated in various β-catenin-related diseases as varied as cancer/proliferative, metabolic, osteoporosis, neurological, immunological, endocrinologic, cardiovascular, hematologic, and diabetes. β-catenin-related disorders are a class of diseases in which there is a dysregulation of the β-catenin signaling pathway, most often with increased cytosolic β-catenin levels that leads to increased translocation to the nucleus and elevated target gene activation.

Human DKK3b is a 38 kDa intracellular regulator of β-catenin signaling and is one of a 4-member family of Wnt regulators that includes DKK1, DKK2, and DKK4. All family members share two 50 to 70 residue long, cysteine-rich domains (N-1 and C-1) that define the family. DKK3b differs from other family members in that it is an intracellular protein, not a secreted polypeptide and does not block Wnt receptor activation. Its related secreted glycoprotein, DKK3, unlike its family members, also cannot block the binding of Wnt to its cognate receptor, Frizzled.

DKK3b is located downstream of the Wnt regulated degradation complex where it regulates β-catenin trafficking to the nucleus and has the capacity to protect β-catenin from proteolysis by blocking ubiquitination and by redirecting it to the actin cytoskeleton. DKK3b rapidly shuttles between the perinuclear space and the cytoplasmic surface of the plasma membrane of astrocytes using myosin motors and actin fibers. DKK3b exerts its regulation on β-catenin and its signaling pathways by capturing β-catenin destined for the nucleus in a complex with β-transducin repeat-containing protein (β-TrCP) that is bound to the actin cytoskeleton and therefore unavailable for nuclear translocation.

DKK3b also acts more broadly to regulate other β-TrCP target substrates that share the degron structure in addition to β-catenin, including NF-kB, p38, Decaptor and Erk/2. NF-kB, p38, and Erk1/2 proteins are also bound to cytoplasmic microfilaments in a stabilized DKK3b-dependent complex. In the case of NF-kB and Decaptor, this sequestration prevents the ubiquitin-based degradation required for activation of the NF-kB and mTOR signaling pathways, and thereby suppresses signaling by both signaling pathways.

As a gatekeeper for β-catenin nuclear entry, DKK3b is an attractive target for the creation of new therapeutic modalities that impact Wnt/β-catenin signaling cascade and expands the therapeutic landscape for intervention in this key pathway for arresting aberrant β-catenin signaling and treating various β-catenin-related diseases. DKK3b and its discovery and its role in regulating β-catenin and the β-catenin signaling pathway is described in WO 2013/148224 and WO 2017/070092. Also described in WO 2013/148224 and WO 2017/070092 are peptide mimetics of DKK3b and variants of DKK3b, with for example, cell signaling peptides and cell penetrating peptides, suitable for recombinant production and suitable for use as therapeutics in the treatment of disease.

The term “peptide mimetics” and “mimetic peptides” have been used interchangeably herein for the description of compounds discovered through a variety of research. A peptide mimetic can be a molecule such as a peptide, a modified peptide or any other molecule that biologically mimics the biological functions of the parent protein or peptide used as a starting reference point for creating the peptide mimetic. Since peptide mimetics are generally engineered to be smaller than the parent molecule, they are often more stable than the parent molecule, easier and less expensive to manufacture, may elicit fewer side effects, and may be engineered to be more potent than the target molecule, just to name a few advantages. While there are some instances in the prior art that the terms “peptide mimetic” and “mimetic peptide” imply a relative size, these terms as used herein should not be considered limiting with respect to the size of the various polypeptide-based molecules referred to herein and which are encompassed within this invention, unless otherwise noted.

A peptide mimetic of DKK3b useful as a therapeutic for the treatment of diseases related to β-catenin dysregulation would be desirable to provide one or more of the many advantages associated with such engineered peptides.

### SUMMARY OF THE INVENTION

The invention provides highly potent and stable peptide mimetics of human DKK3b having numerous improved properties as compared to prior art DKK3b therapeutics. The peptide mimetics of the invention are useful as therapeutics in the treatment of various diseases wherein inhibiting nuclear translocation of β-catenin is therapeutic, including, but not limited to, cancer/proliferative, metabolic, osteoporosis, neurological, immunological, endocrinologic, cardiovascular, hematologic, and inflammatory diseases.

The peptide mimetics comprising an N-terminal domain as described herein, an N-1 domain as described herein, and a C-terminal domain as described herein. Optionally, the peptide mimetic can comprise an amino acid linker between the N-terminal domain and the N-1 domain, and/or an amino acid linker between the N-1 domain and the C-terminal domain.

The peptide mimetics of the invention comprise:
i) an N-terminal domain having an amino acid sequence that comprises a random coil, α-helix, or β-pleated sheet and comprising about 2 to about 3 negatively charged amino acids within the first 6 amino acids of the N-terminal domain;
ii) an N-1 domain that is at least 80% identical to the N-1 domain of human DKK1 having the amino acid sequence of SEQ ID NO: 3, the N-1 domain of human DKK2 having the amino acid sequence of SEQ ID NO: 4, the N-1 domain of human DKK3b having the amino acid sequence of SEQ ID NO: 5, or the N-1 domain of human DKK4 having the amino acid sequence of SEQ ID NO: 6 and wherein the N-1 domain further comprises a cell-penetrating peptide; and
iii) a C-terminal domain having an amino acid sequence comprising a random coil, alpha helix, or β-pleated sheet comprising about 2 to about 3 negatively charged amino acids within the last 6 amino acids of the C-terminal domain; wherein the peptide mimetic is an inhibitor of β-catenin nuclear translocation or a β-catenin signaling pathway. Preferably, the cell-penetrating domain comprises a peptide having about 4 to about 8 amino acids. Preferably, the cell penetrating domain comprises a peptide having about 4 to about 8 Arginine residues. Preferably, wherein one or more cysteine residues of the DKK1, DKK2, DKK3b, or DKK4 N-1 domains are substituted with a conservative amino acid. Preferably, the N-1 domain comprises an amino acid sequence that is at least 80%, at least about 85%, at least about 90%, at least about 95%, at least about 97%, at least about 98%, or at least about 99% identical to one of SEQ ID NOs: 7, 8, 45 and 46. Preferably, the N-1 domain comprises an amino acid sequence that is at least 80%, at least about 85%, at least about 90%, at least about 95%, at least about 97%, at least about 98%, or at least about 99% identical to SEQ ID NO: 45.

In yet additional preferred aspects, the peptide mimetic of the invention comprises:
i) an N-terminal domain having an amino acid sequence that comprises a random coil, α-helix, or β-pleated sheet and comprises about 2 to about 3 negatively charged amino acids within the first 6 amino acids of the N-terminal domain;
ii) an N-1 domain that is a variant of the N-1 domain of human DKK1 (for example, having the amino acid sequence of SEQ ID NO: 3), a variant of the N-1 domain of human DKK2 (for example, having the amino acid sequence of SEQ ID NO: 4), a variant of the N-1 domain of human DKK3b (for example, having the amino acid sequence of SEQ ID NO: 5), or a variant of the N-1 domain of human DKK4 (for example, having the amino acid sequence of SEQ ID NO: 6); wherein the variant comprises a cell-penetrating peptide, and has at least about 75%, at least about 76%, at least about 77%, at least about 78%, at least about 79%, or at least about 80% sequence identity to the N-1 domain of human DKK1, the N-1 domain of human DKK2, the N-1 domain of human DKK3b, or the N-1 domain of human DKK4 (for example, one of SEQ ID NOs: 3, 4, 5 and 6); or
an N-1 domain that has at least about 80% sequence identity to the amino acid sequence of SEQ ID NOs: 7, 8, 45, 46, or 69 and wherein the N-1 domain comprises a cell-penetrating peptide; and
iii) a C-terminal domain having an amino acid sequence comprising a random coil, alpha helix, or β-pleated sheet and comprising about 2 to about 3 negatively charged amino acids within the last 6 amino acids of the C-terminal domain;
   wherein the peptide mimetic is an inhibitor of β-catenin nuclear translocation or a β-catenin signaling pathway. Preferably the cell-penetrating domain is a peptide having about 4 to about 8 amino acids. Preferably the cell penetrating domain is a peptide having about 4 to about 8 Arginine residues. For example, about 4 to about 8 consecutive Arginine residues. Preferably, wherein one or more cysteine residues of the DKK1, DKK2, DKK3b, or DKK4 N-1 domains are substituted with a conservative amino acid. Preferably, the N-1 domain comprises an amino acid sequence that is at least 80%, at least about 85%, at least about 90%, at least about 95%, at least about 97%, at least about 98%, or at least about 99% identical to one of SEQ ID NOs: 7, 8, 45 and 46. Preferably, the N-1 domain comprises an amino acid sequence that is at least 80%, at least about 85%, at least about 90%, at least about 95%, at least about 97%, at least about 98%, or at least about 99% identical to SEQ ID NO: 45.

Preferably, the peptide mimetic comprises an amino acid linker between one or more of the N-terminal domain, N-1 domain and C-terminal domain. The peptide mimetic can comprise an amino acid linker between the N-terminal domain and the N-1 domain, and/or an amino acid linker between the N-1 domain and the C-terminal domain. Preferably, the amino acid linker is about 1 to about 150 amino acids in length. In certain aspects, the peptide mimetic comprises an amino acid linker between the N-terminal domain and the N-1 domain, wherein the amino acid linker is between about 1 and about 70 amino acids in length; or about 1 to about 50 amino acids in length; or about 1 to about 30 amino acids in length; or about 1 to about 20 amino acids in length. In certain additional aspects, the peptide mimetic comprises an amino acid linker between the N-1 domain and the C-terminal domain, wherein the amino acid linker is between about 1 to about 150 amino acids in length; or about 1 to about 100 amino acids in length; or about 1 to about 75 amino acids in length; or about 1 to about 50 amino acids in length; or about 1 to about 30 amino acid in length; or about 1 to about 20 amino acids in length. In certain aspects, the amino acid linkers is about 1 to about 2 amino acids in length.

Preferably, the N-terminal domain comprises negatively charged amino acids at amino acid positions 2, 4 and 5. Preferably, the N-terminal domain comprises or consists of an amino acid sequence that is at least about 80%, at least about 85%, at least about 90%, at least about 95%, at least about 97%, at least about 98%, or at least about 99% identical to SEQ ID NO: 59. Also, preferably, the N-terminal domain comprises or consists of an amino acid sequence that is at least about 80% identical to SEQ ID NO: 48. In yet further aspects, the N-terminal domain comprises or consists of the amino sequence of SEQ ID NO: 59.

Preferably, the C-terminal domain comprises two, consecutive, negatively-charged amino acids within the last 6 amino acids of the C-terminal domain. Preferably, the C-terminal domain comprises two, consecutive, charged amino acids within the last 6 amino acids of the C-terminal domain wherein one charged amino acid is negatively charged and wherein one charged amino acid is positively charge. Preferably, the negatively-charged amino acids are positioned just prior to the last amino acid of the C-terminal domain. Preferably, the two consecutive charged amino acids are positioned just prior to the last amino acid of the C-terminal domain. Preferably, the C-terminal domain comprises or consists of an amino acid sequence that is at least about 80%, at least about 85%, at least about 90%, at least about 95%, at least about 97%, at least about 98%, or at least about 99% identical to SEQ ID NO: 60. Also, preferably, the C-terminal domain comprises or consists of an amino acid sequence that is at least 80% identical to SEQ ID NO: 49. In yet further aspects, the C-terminal domain comprises or consists of the amino acid sequence of SEQ ID NO: 60.

Preferably, the peptide mimetic comprises or consists of the amino acid sequence that is at least about 80%, at least about 85%, at least about 90%, at least about 95%, at least about 97%, at least about 98%,or at least about 99% identical to the amino acid sequence of SEQ ID NO: 1. In yet further aspects, the peptide mimetic comprises or consists of the amino acid sequence of SEQ ID NO: 1. In additional aspects, the peptide mimetic comprises or consists of the amino acid sequence of SEQ ID NO: 70.

In yet additional aspects the peptide mimetic comprises or consists of an amino acid sequence that is at least about 80%, at least about 85%, at least about 90%, at least about 95%, at least about 97%, at least about 98%, or at least about 99% identical to the amino acid sequence of SEQ ID NO: 66, 67 or 68. In yet further aspects, the peptide mimetic comprises the amino acid sequence of SEQ ID NO: 66, 67 or 68.

The invention encompasses a pharmaceutical composition comprising a pharmaceutically acceptable excipient and a peptide mimetic as described herein.

The invention also provides methods of methods of inhibiting nuclear translocation of β-catenin in a patient, the method comprising administering at least one peptide mimetic of the invention to a patient in need thereof. Preferably, the peptide mimetic is delivered as part of a pharmaceutical formulation. Preferably, the pharmaceutical formulation comprises at least one excipient.

Preferably, delivery of at least one peptide mimetic is selected from one or more of subcutaneous delivery, oral delivery, topical delivery, intravitreal delivery, nasal delivery, intravenous delivery, intraarterial delivery, intramuscular delivery, intraperitoneal delivery, and transmucosal delivery.

Preferably, the method is used to treat a disease caused by dysregulation of a β-catenin signaling pathway in a patient. Preferably, the method is used to treat a disease selected from the group of diseases and disorders consisting of cancer/proliferative, metabolic, osteoporosis, neurological, immunological, endocrinologic, cardiovascular, hematologic, and inflammatory diseases.

The invention includes a method of treating cancer in a patient in need thereof comprising administering a peptide mimetic of the invention to a patient in need thereof. Preferably, at least one peptide mimetic is delivered as part of a pharmaceutical formulation, and optionally comprising co-administering a therapeutic anti-cancer treatment regimen.

The invention also includes a method of treating a disease in a patient wherein the disease is treatable by suppressing β-catenin nuclear translocation or a β-catenin pathway comprising administering to the patient, a therapeutically effective amount of a peptide mimetic described herein. For example, the disease can be selected from the group of diseases and disorders selected from the group consisting of cancer/proliferative, metabolic, osteoporosis, neurological, immunological, endocrinologic, cardiovascular, hematologic, and inflammatory.

Preferably, the peptide mimetic used in the methods described herein has an amino acid sequence having at least about 80%, at least about 85%, at least about 90%, at least about 95%, at least about 97%, at least about 98%, or at least about 99% identity to an amino acid sequence of SEQ ID NO: 1. Preferably, the peptide comprises the amino acid sequence of SEQ ID NO: 1, 66, 67 and 68. Preferably, the method is used to treat a disease caused by dysregulation of a β-catenin signaling pathway in a patient. Preferably, the methods of the invention further comprise co-administering a therapeutic anti-cancer treatment regimen.

The invention also includes a nucleic acid molecule encoding a peptide mimetic described herein as well as a vector or host cell comprising the nucleic acid molecule. In yet further aspects, the host cell comprising a vector comprising a nucleic acid molecule encoding a peptide mimetic described herein. The host cell can be selected from the group consisting of mammalian cells, bacterial cells, yeast cells, insect cells and plant cells. In certain specific aspects, the nucleic acid molecule encodes a peptide mimetic comprising an amino acid sequence of SEQ ID NOs: 1, 66, 67, 68 and 70. The vector can, for example, be a viral vector, such as lentivirus, retrovirus, adenovirus and Adeno-associated viruses. The host cell can be a mammalian cell, for example, a mammalian cell expression system comprising a vector described herein; the mammalian cell expression system can for example selected from CHO cells and HEK293 cells.

The invention additionally includes a method for the preparation of a peptide mimetic by chemical synthetic methods as well as a peptide mimetic prepared by such methods.

In certain aspects, the peptide mimetic can further comprise a purification tag. For example, the purification tag can be a poly-histidine (His) tag, a cMyc tag, or a FLAG tag. The invention also includes a peptide mimetic further comprising a signal secretion peptide; for example, the signal secretion peptide is selected from the IL-2 signal peptide, the IgG signal peptide, the Igkappa signal peptide and the azuricidin signal peptide.

### BRIEF DESCRIPTION OF THE DRAWINGS

FIG. 1 is a schematic of an annotated sequence of human wild type (wt) DKK3b showing the amino acid location of the N-terminal domain, the N-1 domain, the putative Loop 2 of the N-1 domain and the C-terminal domain.

FIG. 2 is a graph showing a comparison of the B-catenin silencing activity between AC1 (SEQ ID NO: 1) and cpDKK3b (SEQ ID NO: 2) in TopFlash Reporter cells.

FIG. 3 is a graph showing a comparison of the β-catenin silencing activity between AC1 (SEQ ID NO: 1) and cpDKK3b (SEQ ID NO: 2) in ovarian cancer cells (OVCAR3) and colorectal cancer cells (Colo205).

FIG. 4 is a graph showing bioavailability of AC1 (SEQ ID NO: 1) in nude mice harboring xenograft tumors of OVCAR3 cells.

FIG. 5 is a graph showing tumor beta-catenin signaling (as percentage of untreated control) over time (days after injection) for mice implanted with ovarian cancer (OVCAR3) cells expressing a beta catenin dependent luciferase cDNA and treated with 1 μg of AC2.

### DETAILED DESCRIPTION OF THE INVENTION

Definitions

The term “amino acid(s)”, or when associated with a peptide, “amino acid residue(s)” or “residue(s)” as used interchangeably herein includes all naturally occurring amino acids. Amino acids are classified as hydrophobic, polar and charged. Table 1 shows the classification of the most common amino acids and will be referenced herein when describing the peptide mimetic of the invention.

TABLE 1

| | | |
|---|---|---|
| Amino Acid | Three letter code | Single letter code |

Nonpolar Amino Acids (hydrophobic)

| | | |
|---|---|---|
| glycine | Gly | G |
| alanine | Ala | A |
| valine | Val | V |
| leucine | Leu | L |
| isoleucine | Ile | I |
| methionine | Met | M |
| phenylalanine | Phe | F |
| tryptophan | Trp | W |
| proline | Pro | P |

Polar (hydrophilic)

| | | |
|---|---|---|
| serine | Ser | S |
| threonine | Thr | T |
| cysteine | Cys | C |
| tyrosine | Tyr | Y |
| asparagine | Asn | N |
| glutamine | Gln | Q |

Electrically Charged (Negative)

| | | |
|---|---|---|
| aspartic acid | Asp | D |
| glutamic acid | Glu | E |

Electrically Charged (Positive)

| | | |
|---|---|---|
| lysine | Lys | K |
| arginine | Arg | R |
| histidine | His | H |

Amino acids other than those found in Table 1 may be used in the peptide mimetics of the invention. For example, amino acids modified by natural processes such as by post-translation processing or by chemical modification techniques which are well known in the art.

As used herein the phrase “a peptide mimetic of a protein inhibitor of β-catenin nuclear translocation” refers to a rationally designed peptide of the invention capable of specifically binding β-catenin destined for the nucleus in a complex with β-transducin repeat-containing protein (β-TrCP) and thereby rendering the β-catenin unavailable for nuclear translocation.

As used herein, the phrase “a peptide mimetic of a protein inhibitor of a β-catenin pathway” refers to a rationally designed peptide of the invention capable of specifically interacting with a component of a β-catenin pathway, resulting in reduction or inhibition of the effect of β-catenin on a biological system. As used herein a “β-catenin pathway” refers to any signal transduction pathways upstream or downstream of β-catenin (e.g., including the Wnt/β-catenin signaling pathway). The inhibition of a β-catenin pathway by a peptide mimetic of the invention preferably inhibits β-catenin directly by targeting its capture in a multi component complex that prevents nuclear translocation and thereby, silences β-catenin directed gene expression.

“Secretion recognition peptides” (SRP) also referred to herein as a “signal secretion peptides” include any peptide capable of engaging the SRP receptor (translocon) in the ER membrane required to move the growing polypeptide chain across the ER membrane for secretion.

As used herein the term “specifically binds”, “specifically recognizes” or “specifically interacts with” when referring to a peptide mimetic of a protein inhibitor of β-catenin nuclear translocation, refers to a peptide mimetic which preferentially locks the degron of β-catenin to the WD40 domain of β-TrCP, preferably with high affinity as compared to, for example, other biological molecules present in a cell in which the peptide mimetic has been introduced for the purposes of inhibition of β-catenin or a β-catenin pathway. Likewise, a peptide mimetic of the invention may also preferentially bind with high affinity a binding partner or other target in a β-catenin pathway for indirect inhibit a β-catenin. Preferably, the peptide mimetics of the invention should ideally specifically bind target molecules of human wild type DKK3b protein. By this is meant that the peptide mimetic of human DKK3b should ideally bind target molecules such as β-catenin with similar affinity or higher affinity as compared to the affinity at which human wild type DKK3b protein binds it targets, but should not to any significant degree bind to molecules that the wild type DKK3b does not bind to.

“Human wild type DKK3b” may also be referred to herein as “wthDKK3b” “hDKK3b” or “DKK3b”. Human wild type DKK3b has the amino acid sequence of SEQ ID NO: 47. As used herein the “N-terminal domain of hDKK3b” comprises amino acid ranging from 1 to about 20 or 21 of SEQ ID NO: 47. As used herein the C-terminal domain of hDKK3b comprises amino acids ranging from about 266 to about 279 of SEQ ID NO: 47. As used herein the N-1 domain of hDKK3 comprises amino acids ranging from about 74 to 126.

A “conservative” amino acid substitution refers to the substitution of an amino acid in a polypeptide with another amino acid having similar properties, such as size or charge. In certain embodiments, a polypeptide comprising a conservative amino acid substitution maintains at least one activity of the unsubstituted polypeptide.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term pharmaceutical composition” refers to the combination of an active agent (e.g., binding agent) with a carrier, inert or active, making the composition especially suitable for diagnostic or therapeutic use in vitro, in vivo or ex vivo.

As used herein, a “diagnostic” or “diagnostic test” includes the detection, identification, or characterization of a disease state or condition of a subject. For example, a disease or condition may be characterized to determine the likelihood that a subject with a disease or condition will respond to a particular therapy, determine the prognosis of a subject with a disease or condition (or its likely progression or regression), determine the effect of a treatment on a subject with a disease or condition, or determine a future treatment course of action.

The term “efficacy” of a treatment or method according to the invention can be measured based on changes in the course of disease or condition in response to a use or a method according to the invention. For example, the efficacy of a treatment or method according to the invention can be measured by its impact on i) different relevant clinical endpoints and/or on ii) surrogate markers such as the impact of the therapeutic compounds in different animal or in vitro systems.

The term “effective amount” as used herein refers to an amount of at least one mimetic peptide according to the invention, or a pharmaceutical formulation thereof, that elicits a detectable reduction of the symptoms of the disease being treated in a subject that is being administered a peptide mimetic of the invention.

As used herein, “inhibiting,” or “ inhibition” or “to inhibit” in the context of “inhibiting β-catenin nuclear translocation” or “inhibiting a β-catenin signaling pathway” generally means either reducing, decreasing, suppressing, blocking, or antagonizing the activity of a target. In particular, “modulating” or “to modulate” can mean either reducing or suppressing the activity of, a (relevant or intended) biological activity (e.g., β-catenin nuclear translocation) of a target (e.g., β-catenin), by at least 5%, at least 10%, at least 25%, at least 50%, at least 60%, at least 70%, at least 80%, or 90% or more, compared to activity of the target in the same assay under the same conditions but without the presence of the peptide mimetic of the invention, i.e., baseline.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintegrates (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975).

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families (see above). The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position. Preferably the comparison window is a contiguous stretch of the reference sequence of about and preferably from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 150 amino acids, from about 150 amino acids to about 155 amino acids, from about 155 amino acids up to the full-length of the reference sequence. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software.

As used herein, the term “subject” or “patient” refers to any organism to which a composition in accordance with the present disclosure may be administered, e.g., for experimental, diagnostic, prophylactic, and/or therapeutic purposes. Typical subjects include animals (e.g., mammals such as mice, rats, rabbits, non-human primates, and humans) and/or plants. Preferably “patient” refers to a human subject who may seek or be in need of treatment, requires treatment, is receiving treatment, will receive treatment, or a subject who is under care by a trained professional for a particular disease or condition.

As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

The term “protein” or “peptide” as used herein refers to a at least two or more amino acid residues linked together by peptide bond. The amino acid sequence in a protein or peptide is shown in the standard format, i.e., from amino terminus (N-terminus) to carboxyl terminus (C-terminus).

As used herein, the term “therapeutically effective amount” means an amount of an agent to be delivered (e.g., nucleic acid, protein or peptide, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. For example, in cancer or pathologies related to unregulated cell division, a therapeutically effective amount refers to that amount which has the effect of (1) reducing the size of a tumor, (2) inhibiting (that is, slowing to some extent, preferably stopping) aberrant cell division, for example cancer cell division, (3) inducing cancer or tumor cell death and/or inhibiting cancer or tumor cell proliferation, (4) preventing or reducing the metastasis of cancer cells, and/or, (5) relieving to some extent (or, preferably, eliminating) one or more symptoms associated with a pathology related to or caused in part by unregulated or aberrant cellular division, including for example, cancer, or angiogenesis.

The terms “treating” or “treatment” of a disease (or a condition or a disorder) as used herein refer to preventing the disease from occurring in a human subject or an animal subject that may be predisposed to the disease but does not yet experience or exhibit symptoms of the disease (prophylactic treatment), inhibiting the disease (slowing or arresting its development), providing relief from the symptoms or side-effects of the disease (including palliative treatment), and causing regression of the disease. For example, with regard to cancer, these terms also mean that the life expectancy of an individual affected with a cancer may be increased or that one or more of the symptoms of the disease will be reduced. For example, with regard to cancer, “treating” also includes enhancing or prolonging an anti-tumor response in a subject.

As used herein any form of administration or coadministration of a “combination”, “combined therapy” and/or “combined treatment regimen” refers to treatment of a disease with at least two therapeutically active drugs or compositions which may be administered or co-administered”, simultaneously, in either separate or combined formulations, or sequentially at different times separated by minutes, hours or days, but in some way act together to provide the desired therapeutic response.

As used herein, the term “about” or “approximately” as applied to one or more values of interest, refers to a value that is similar to a stated reference value. In certain embodiments, the term “approximately” or “about” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “variant” refers to molecules which differ in their amino acid sequence from a native or reference sequence. The amino acid sequence variants may possess substitutions, deletions, and/or insertions at certain positions within the amino acid sequence, as compared to a native or reference sequence. Substantially homologous means a variant amino acid sequence which is identical to the referenced peptide sequence except for the deletion, insertion and/or substitution of 1, 2, 3, 4, 5 or 6 amino acid residues. The identity of two amino acid sequences can be determined by visual inspection and/or mathematical calculation, or more easily by comparing sequence information using known computer programs used for sequence comparison. A variant may comprise a sequence having at least one conservatively substituted amino acid, meaning that a given amino acid residue is replaced by a residue having similar physiochemical characteristics. Desired amino acid substitutions (whether conservative or non-conservative) can be determined by those skilled in the art at the time such substitutions are desired. Preferably, variants will possess at least about 50% identity (homology) to a native or reference sequence, and preferably, they will be at least about 75%, at least about 80%, more preferably at least about 90% identical (homologous) to a native or reference sequence.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

As used herein, the term “recombinant” generally refers to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc.). Recombinant also refers to the polypeptide encoded by the recombinant nucleic acid. Non-naturally occurring nucleic acids or polypeptides include nucleic acids and polypeptides modified by man.

As used herein, “fusion protein” refers to a protein formed from the combination of at least two different proteins or protein fragments. A fusion protein is encoded by a recombinant DNA molecule.

Table A below lists some of the amino acid sequences referred to herein as well as their corresponding SEQ ID NO, wherein each of ϕ₁, ϕ₂, and ω are as defined below:

| SEQ ID | |
|---|---|
| NO: | AMINO ACID SEQUENCE |
| 1 | MDAEDLLLKLNLAATVGTAPPKGKNLGQAYPASSDKESEVGRYSHSPHQ |
| | GSSACMVARRRRRRAHRDGMACPSTRSNNGIAIPVPTAALLIILGGDDI |
| 2 | MRGSLKHHHHHHLKGMASMTGGQQMKLGDIYARAAARQARADIGGST |
| | MEAEEAAAKASSEVNLANLPPSYHNETNTDTKVGNNTIHVHREIHKITNN |
| | QTGMVFSETVITSVGDEEGRRSHECIIDEDCGPSMYCQFASFQYTCQPCRG |
| | QRMLCTRDSECCGDQLCVWGHCTKMATRGSNGTICDNQRDCQPGLCCA |
| | FQRGLLFPVCTPLPVEGELCHDPASRLLDLITWELEPDGALDRCPCASGLL |
| | CQPHSHSLVYVCKPTFVGSRDQDGEILLPREVPDEYEVGSFMEEVRQELE |
| | DLERSLTEEMALREPAAAAAALLGGEEI |
| 3 | QTIDNYQPYPCAEDEECGTDEYCASPTRGGDAGVQICLACRKRRKRCMR |
| | HAM |
| | CCPGNYCKNGICVSS |
| 4 | CSSDKECEVGRYCHSPHQGSSACMVCRRKKKRCHRDGMCCPSTRCNNGI |
| | CIPV |
| 5 | HECIIDEDCGPSMYCQFASFQYTCQPCRGQRMLCTRDSECCGDQLCVWG |
| | HCTK |
| 6 | DLHGARKGSQCLSDTDCNTRKFCLQPRDEKPFCATCRGLRRRCQRDAMC |
| | CPGTLCVNDVCT |
| 7 | HEAIIDEDSGPSMYSQFASFQYTCQPARRRRRRATRDSESCGDQLAVWGH |
| | STK |
| 8 | QTIDNYQPYPAAEDEESGTDEYSASPTRGGDAGVQICLAARRRRRRAMRH |
| | AMSCPGNYAKNGIAVSS |
| 25 | RRRRRR(R)₁₋₆ |
| 45 | KGKNLGQAYPASSDKESEVGRYSHSPHQGSSACMVARRRRRRAHRDGM |
| | ACPSTRSNNGIAIPV |
| 46 | DLHGARKGSQALSDTDSNTRKFSLQPRDEKPFCATARRRRRRAQRDAMS |
| | CPGTLSVNDVAT |
| 47 | MEAEEAAAKASSEVNLANLPPSYHNETNTDTKVGNNTIHVHREIHKITNN |
| | QTGQMVFSETVITSVGDEEGRRSHECIIDEDCGPSMYCQFASFQYTCQPCR |
| | GQRMLCTRDSECCGDQLCVWGHCTKMATRGSNGTICDNQRDCQPGLCC |
| | AFQRGLLFPVCTPLPVEGELCHDPASRLLDLITWELEPDGALDRCPCASGL |
| | LCQPHSHSLVYVCKPTFVGSRDQDGEILLPREVPDEYEVGSFMEEVRQELE |
| | DLERSLTEEMALREPAAAAAALLGGEEI |
| 48 | MEADELLLKLNLAATVGFAPP |
| 49 | TSQLLIILGGDDI |
| 50 | MTRLTVLALLAGLLASSRAGSGRGHHHHHHVGTGSNSPGMDAEDLLLKL |
| | NLAATVGTAPPKGKNLGQAYPASSDKESEVGRYSHSPHQGSSACMVARR |
| | RRRRAHRDGMACPSTRSNNGIAIPVPTAALLIILGGDDI |
| 54 | CLACRKRRKRCMRHMCC |
| 55 | CMVCRRKKKRCHRDGMCC |
| 56 | CQPCRGQRMLCTRDSECC |
| 57 | CATCRGLRRRCQRDAMCC |
| 58 | MRGSLKHHHHHHLKGMASMTGGQQMKLGDIYARAAARQARADIGGST |
| | MEAEEAAAKASSEVNLANLPPSYHNETNTDTKVGNNTIHVHREIHKITNN |
| | QTGQMVFSETVITSVGDEEGRRSHECIIDEDCGPSMYCQFASFQYTCQPCR |
| | GQRMLCTRDSECCGDQLCVWGHCTKMATRGSNGTICDNQRDCQPGLCC |
| | AFQRGLLFPVCTPLPVEGELCHDPASRLLDLITWELEPDGALDRCPCASGL |
| | LCQPHSHSLVYVCKPTFVGSRDQDGEILLPREVPDEYEVGSFMEEVRQELE |
| | DLERSLTEEMALREPAAAAAALLGGEEI |
| 59 | MDAEDLLLKLNLAATVGTAPP |
| 60 | TAALLIILGGDDI |
| 61 | DAEDLLLKLNLAATVGTAPP |
| 62 | _{ϕ1}DAEDLLLKLNLAATVGTAPP |
| 63 | ADAEDLLLKLNLAATVGTAPP |
| 64 | IDAEDLLLKLNLAATVGTAPP |
| 65 | DAEDLLLKLNLAATVGTAPPKGKNLGQAYPASSDKESEVGRYSHSPHQGS |
| | SACMVARRRRRRAHRDGMACPSTRSNNGIAIPVPTAALLIILGGDDI |
| 66 | _{ϕ2}DAEDLLLKLNLAATVGTAPPKGKNLGQAYPASSDKESEVGRYSHSPHQ |
| | GSSACMVARRRRRRAHRDGMACPSTRSNNGIAIPVPTAALLIILGGDDI |
| 67 | ADAEDLLLKLNLAATVGTAPPKGKNLGQAYPASSDKESEVGRYSHSPHQ |
| | GSSACMVARRRRRRAHRDGMACPSTRSNNGIAIPVPTAALLIILGGDDI |
| 68 | IDAEDLLLKLNLAATVGTAPPKGKNLGQAYPASSDKESEVGRYSHSPHQG |
| | SSACMVARRRRRRAHRDGMACPSTRSNNGIAIPVPTAALLIILGGDDI |
| 69 | ASSDKESEVGRYSHSPHQGSSACMVARRRRRRAHRDGMACPSTRSNNGI |
| | AIPV |
| 70 | ϕ₂DAEDLLLKLNLAATVGTAPPKGKNLGQAYPASSDKESEVGRYSHSPHQ |
| | GSSAωMVARRRRRRAHRDGMAωPSTRSNNGIAIPVPTAALLIILGGDDI |
| 71 | ADAEDLLLKLNLAATVGTAPPKGKNLGQAYPASSDKESEVGRYSHSPHQ |
| | GSSAωMVARRRRRRAHRDGMAωPSTRSNNGIAIPVPTAALLIILGGDDI |
| 72 | IDAEDLLLKLNLAATVGTAPPKGKNLGQAYPASSDKESEVGRYSHSPHQG |
| | SSAωMVARRRRRRAHRDGMAωPSTRSNNGIAIPVPTAALLIILGGDDI |
| 73 | CSSDKECEVGRYCHSPHQGSSACMVCRRRRRRCHRDGMCCPSTRCNNGI |
| | CIPV |
| 74 | ASSDKESEVGRYSHSPHQGSSAωMVARRRRRRAHRDGMAωPSTRSNNGI |
| | AIPV |
| 75 | IDAEDLLLKLNLAATVGTAPPKGKNLGQAYPASSDKESEVGRYSHSPHQG |
| | SSASMVARRRRRRAHRDGMAAPSTRSNNGIAIPVPTAALLIILGGDDI |
| 76 | IDAEDLLLKLNLAATVGTAPPKGKNLGQAYPASSDKESEVGRYSHSPHQG |
| | SSAAMVARRRRRRAHRDGMASPSTRSNNGIAIPVPTAALLIILGGDDI |
| 77 | ADAEDLLLKLNLAATVGTAPPKGKNLGQAYPASSDKESEVGRYSHSPHQ |
| | GSSASMVARRRRRRAHRDGMAAPSTRSNNGIAIPVPTAALLIILGGDDI |
| 78 | ADAEDLLLKLNLAATVGTAPPKGKNLGQAYPASSDKESEVGRYSHSPHQ |
| | GSSAAMVARRRRRRAHRDGMASPSTRSNNGIAIPVPTAALLIILGGDDI |
| 79 | MDAEDLLLKLNLAATVGTAPPKGKNLGQAYPASSDKESEVGRYSHSPHQ |
| | GSSASMVARRRRRRAHRDGMAAPSTRSNNGIAIPVPTAALLIILGGDDI |
| 80 | MDAEDLLLKLNLAATVGTAPPKGKNLGQAYPASSDKESEVGRYSHSPHQ |
| | GSSAAMVARRRRRRAHRDGMASPSTRSNNGIAIPVPTAALLIILGGDDI |

### Peptide Mimetics

The present invention provides peptide mimetics of DKK3b, a protein inhibitor of β-catenin nuclear translocation and of a β-catenin signaling pathway. The peptides of the invention are engineered peptides that mimic the function of human wild type DKK3b (also referred to herein as “wtDKK3b” or “hDKK3b”). Wild type human DKK3b has the amino acid sequence of SEQ ID NO: 47:

(SEQ ID NO: 47)
MEAEEAAAKASSEVNLANLPPSYHNETNTDTKVGNNTIHVHREIHKITNN
QTGQMVFSETVITSVGDEEGRRSHECIIDEDCGPSMYCQFASFQYTCQPC
RGQRMLCTRDSECCGDQLCVWGHCTKMATRGSNGTICDNQRDCQPGLCCA
FQRGLLFPVCTPLPVEGELCHDPASRLLDLITWELEPDGALDRCPCASGL
LCQPHSHSLVYVCKPTFVGSRDQDGEILLPREVPDEYEVGSFMEEVRQEL
EDLERSLTEEMALREPAAAAAALLGGEEI

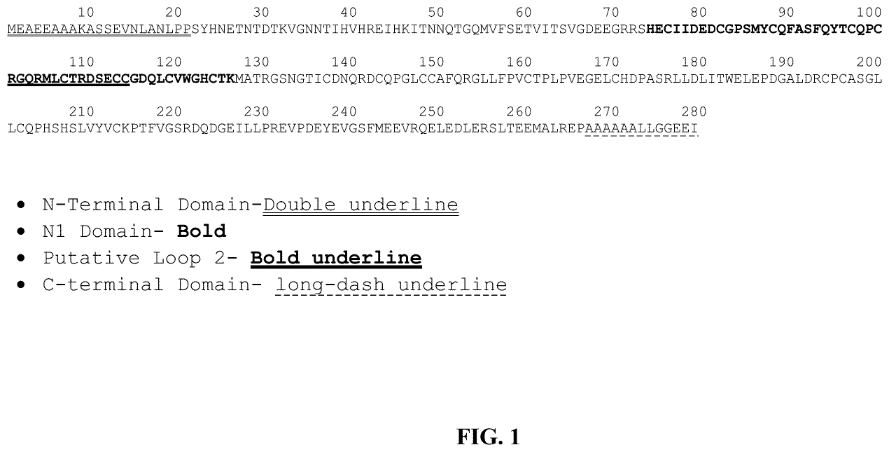

FIG. 1 is schematic of the human wtDKK3b including the N-terminal domain, the C-terminal domain, the N-1 domain and the Loop 2 region of the N-1 domain.

DKK3b was previously discovered as a novel intracellular member of the DKK protein family. Previous studies showed that DKK3b specifically binds the E3 ubiquitin ligase component, β-transducin repeat containing protein β-TrCP, and that this complex in turn binds unphosphorylated β-catenin thereby preventing its nuclear import. Based on this discovery human DKK3b performs at least two roles, 1) β-catenin sequestration; and 2) β-catenin translocation; n.

In previous studies, DKK3b′s potential as a cancer therapeutic was tested. Human DKK3b with cell penetrating capability was generated for therapeutic delivery by fusing a cell penetrating (cp) peptide to the N-terminus of DKK3b, as well as a poly-His tag and recombinantly producing the fusion protein in bacteria. The resulting fusion protein is referred to herein as “cpDKK3b”. The cpDKK3b fusion protein has the amino acid sequence of SEQ ID NO: 58:

(SEQ ID NO: 58)
MRGSLKHHHHHHLKGMASMTGGQQMKLGDIYARAAARQARADIGGSTMEA
EEAAAKASSEVNLANLPPSYHNETNTDTKVGNNTIHVHREIHKITNNQTG
QMVFSETVITSVGDEEGRRSHECIIDEDCGPSMYCQFASFQYTCQPCRGQ
RMLCTRDSECCGDQLCVWGHCTKMATRGSNGTICDNQRDCQPGLCCAFQR
GLLFPVCTPLPVEGELCHDPASRLLDLITWELEPDGALDRCPCASGLLCQ
PHSHSLVYVCKPTFVGSRDQDGEILLPREVPDEYEVGSFMEEVRQELEDL
ERSLTEEMALREPAAAAAALLGGEEI.

An additional fusion protein is represented by SEQ ID NO: 2:

(SEQ ID NO: 2)
MRGSLKHHHHHHLKGMASMTGGQQMKLGDIYARAAARQARADIGGSTMEA
EEAAAKASSEVNLANLPPSYHNETNTDTKVGNNTIHVHREIHKITNNQTG
MVFSETVITSVGDEEGRRSHECIIDEDCGPSMYCQFASFQYTCQPCRGQR
MLCTRDSECCGDQLCVWGHCTKMATRGSNGTICDNQRDCQPGLCCAFQRG
LLFPVCTPLPVEGELCHDPASRLLDLITWELEPDGALDRCPCASGLLCQP
HSHSLVYVCKPTFVGSRDQDGEILLPREVPDEYEVGSFMEEVRQELEDLE
RSLTEEMALREPAAAAAALLGGEEI.

Purification of the unfolded protein produces a linear polypeptide chain that when added to cells in vitro or injected into tumor bearing mice in vivo, promptly and selectively arrests cancer cell proliferation and rapidly initiates tumor cell apoptosis (WO 2013/148224; the contents of which are expressly incorporated herein). Importantly, the cpDKK3b fusion protein did not appear to have any adverse effects in mice when given twice daily for 35 days.

Follow on studies (WO2017/070092; the contents of which are expressly incorporated herein) focused on optimizing human DKK3b for therapeutic use by designing a variant of DKK3b that had improved features including improved cell penetrating peptides (cp) and secretion recognition peptides (SRP). Based on the data and information from those studies, several additional variants were produced using various cell penetrating peptides and SRPs and tested.

Those studies found that the N-terminal 122 amino acids of human wtDKK3b and the last 10 residues at the C-terminus harbored domain(s) that were essential for inhibition of β-catenin translocation to the nucleus and associated tumor suppressor function. Fusion of the N-terminal 122 amino acids to the last 10 residues of the C-terminus produced a fully functional tumor suppressor. Further analysis revealed that residues between amino acid 12 to amino acid 70 of human wild type DKK3b are also not required for inhibition of β-catenin translocation and associated tumor suppressor activity.

The present inventor discovered that the previous DKK3b fusion proteins and variants could be significantly improved upon through engineering of a peptide mimetic of DKK3b capable of inhibiting β-catenin nuclear translocation or capable of inhibiting a β-catenin signaling pathway. The engineered peptide mimetics of the invention having the inhibitory functionality of DKK3b provides additional improvements and enhancements over, for example the cpDKK3b (SEQ ID NO: 2) and variants thereof previously described. The protein mimetics of the invention are superior as compared to, for example, cpDKK3b and variants thereof because they can be engineered to eliminate unnecessary sequence, reduce or eliminate antigenicity, improve and streamline recombinant production of the protein mimetic, increase membrane permeability and cell signaling, increase stability, increase biological half-life and/or increase potency as an inhibitor of β-catenin or as an inhibitor of a β-catenin pathway.

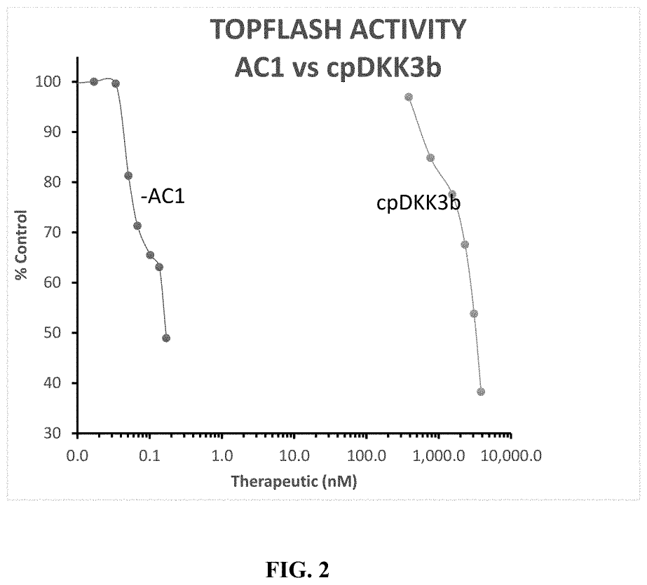

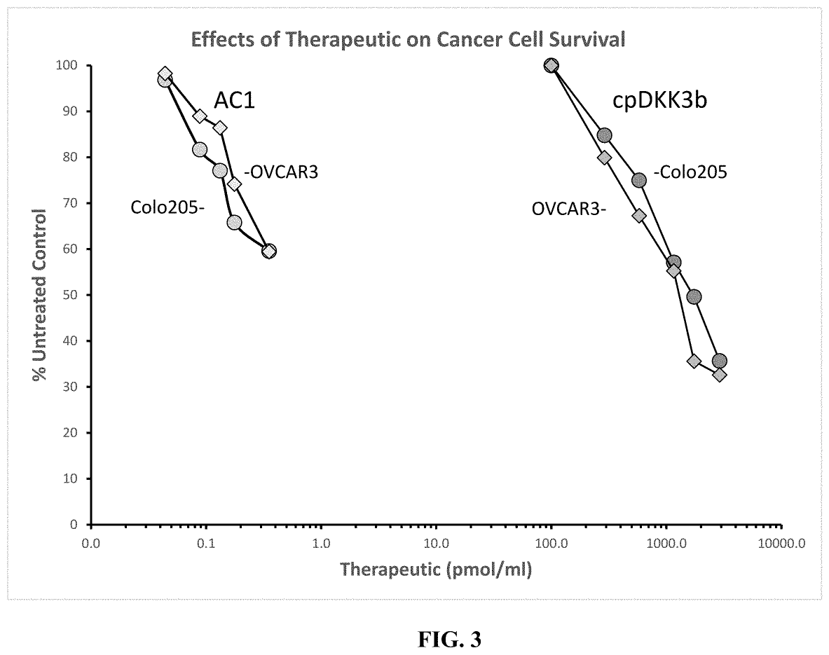

For example, the peptide mimetics of the invention also provide a protein that has a folded confirmation as compared to, for example, the denatured and linear cpDKK3b fusion protein. The potency of the peptide mimetics of the invention is up to 10,000 fold greater than that of cpDKK3b (FIGS. 2 and 3).

The peptide mimetics in accordance with the invention have been engineered to include the following features:
1) elimination of an exogenous cell penetrating cp peptide of cpDKK3b;
2) replacement of the N- and C-terminal domains of cpDKK3b with random amino acids that form a random coil, α-helix, or β-pleated sheet and contain from about 2 to about 3 negatively charged residues within the first about 6 amino acids of the N-terminal domain and from about 2 to about 3 negatively charged amino acids within the last 6 amino acids of the C-Terminal domain;
3) Elimination of all other domains of wtDKK3b except for the N-1 domain;
4) Optional substitution of the cpDKK3b N-1 domain with the N-1 domain of any one of DKK1, DKK2, or DKK4;
5) Modifying the chosen N-1 domain to include a cell penetrating peptide;
6) Optionally modifying the N-1 domain to remove one or more cysteine residues; and
7) Optional linkers of about 1 to about 150, about 1 to about 100, about 1 to about 75, about 1 to about 50, about 1 to about 30, about 1 to about 20 amino acids and preferably linkers of about 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 75, 80, 90, 100, 110, 120, 130, 140, or 150 amino acids between one or more of the domains of the peptide mimetic and preferably those linkers at the region of the N-terminal or C-terminal domains comprise a secondary structure that facilitates the formation of a random coil, α-helix, or β-pleated sheet.

Elimination of an exogenous cell penetrating peptides removes a potential antigenic target in the engineered protein mimetic of DKK3b. It was discovered that the cell penetrating peptide could instead be incorporated into N-1 domain of the protein mimetic as discussed below.

Replacement of the N-terminal 66 amino acids of cpDKK3b (amino acids 1-66 of SEQ ID NO: 58) with preferably about 19 to about 22 random amino acids eliminates glycosylation sites within the peptide mimetic. The present inventor has discovered that glycosylation of any human DKK3b protein or variant thereof as well as any engineered peptide mimetic of DKK3b inactivates its function as an inhibitor of β-catenin nuclear translocation or of a β-catenin signaling pathway. This is particularly important for peptide mimetics produced by recombinant techniques in cells lines such as mammalian cell lines in which post-translational glycosylation takes place. The elimination of post translational glycosylation by the secretory machinery of the cells allows for the production of a peptide mimetic in any desirable cell system including mammalian cells.

The functionality of the N-terminal domain of the peptide mimetic may be generalized as compared to cpDKK3b. Throughout this disclosure, reference is made to the amino acid positions of the N-terminal domain. In most peptides produced using cellular expression systems, including mammalian recombinant systems, the amino acid at position 1 of a protein is methionine. In certain bacterial systems, the amino acid at position 1 is N-formylmethionine (fMet). In peptides produced by chemical peptide synthesis (including, for example, solution and solid phase chemical peptide synthesis), the N-terminus may lack the starting methionine and/or be replaced by another amino acid. The N-terminal domains encompassed by the present invention include an N-terminal domain where the amino acid at position 1 is methionine as well as an N-terminal domain where the amino acid position at position 1 is not methionine. In certain aspects, the peptides and peptide mimetics described herein comprise an N-terminal domain wherein the amino acid at position 1 of the N-terminal domain is serine, threonine, or a nonpolar amino acid other than proline. In additional aspects, the amino acid at position 1 of the N-terminal domain is methionine. In certain additional aspects, position 1 of the N-terminal domain is alanine or isoleucine. The amino acid at position 1 of the N-terminal domain and of the peptide mimetic can be also be methionine, fMet, or serine, threonine or a nonpolar amino acid other than proline and methionine, including, for example, alanine, and isoleucine. The amino acid sequence of the N-terminal domain may be random after the starting amino acid at position 1, for example, after the starting methionine at position 1, of the N-terminal domain, so long as the random amino acid sequence forms a random coil, α-helix, or β-pleated sheet and includes at least two or three negatively charged amino acids within the first 6 amino acids of the N-terminal domain. Preferably, the amino acids that form the random coil, α-helix, or β-pleated sheet do not include proline as proline is unable to stabilize a helix or β-pleated sheet. This strategy may further be optimized by positioning at least one of the negatively charged amino acids just after the amino acid at position 1, for example, the starting Methionine, of the N-terminal sequence. This strategy may be further optimized by positioning negatively charged amino acids at positions 2, 4 and 5 of the N-terminal domain.

A preferred N-terminal domain comprises or consists of the following amino acid sequence: MDAEDLLLKLNLAATVGTAPP (SEQ ID NO: 59). In yet further aspects, the N-terminal domain consists of SEQ ID NO: 59. Another exemplary N-terminal domain has the following amino acid sequence: MEADELLLKLNLAATVGFAPP (SEQ ID NO: 48). Variants SEQ ID NO: 48 or SEQ ID NO: 59 with one or more amino acid additions, deletions, and/or conservative substitutions that preserve the inhibitory activity on β-catenin nuclear translocation are also contemplated. The effects of the amino acid addition(s), deletion(s), and/or substitution(s) on the activity of any variant can be tested using routine methods and assays known in the art and described in the examples. Preferably, peptide mimetics of the invention can include an N-terminal domain having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity or similarity to the sequences of SEQ ID NO: 59. Preferably, peptide mimetics of the invention can include an N-terminal domain having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity or similarity to the sequences of SEQ ID NO: 59 over a contiguous stretch of about 20 amino acids up to the full length of SEQ ID NO: 59. In additional aspects, peptide mimetics of the invention can include an N-terminal domain having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity or similarity to the sequences of SEQ ID NO: 48. In further embodiments, peptide mimetics of the invention can include an N-terminal domain having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity or similarity to the sequences of SEQ ID NO: 48 over a contiguous stretch of about 20 amino acids up to the full length of SEQ ID NO: 48.

Another preferred N-terminal domain comprises the following amino acid sequence: DAEDLLLKLNLAATVGTAPP (SEQ ID NO: 61) (starting from the second amino acid). In yet further aspects, the N-terminal domain comprises or consists of the following amino acid sequence: Φ₁DAEDLLLKLNLAATVGTAPP (SEQ ID NO: 62), wherein Φ₁ is a nonpolar amino acid other than proline (including, for example, methionine, alanine, and isoleucine), or is serine or threonine; in certain aspects, Φ₁ is threonine, serine or a nonpolar amino acid other than proline and methionine. In additional aspects, Φ₁ is alanine or isoleucine. In yet further aspects, the N-terminal domain consists of SEQ ID NO: 62. In certain preferred aspects, the N-terminal domain comprises or consists of one of the following amino acid sequences: ADAEDLLLKLNLAATVGTAPP (SEQ ID NO: 63) or IDAEDLLLKLNLAATVGTAPP (SEQ ID NO: 64). Variants of any one of SEQ ID NOs: 61, 62, 63 and 64 with one or more amino acid additions, deletions, and/or conservative substitutions that preserve the inhibitory activity on β-catenin nuclear translocation are also contemplated and can be used as the N-terminal domain. The effects of the amino acid addition(s), deletion(s), and/or substitution(s) on the activity of any variant can be tested using routine methods and assays known in the art and described in the examples. Preferably peptide mimetics of the invention can include an N-terminal domain having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity or similarity to the sequences of any one of SEQ ID NOs: 61, 62, 63 and 64. Preferably peptide mimetics of the invention can include an N-terminal domain having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity or similarity to the sequences of any one of SEQ ID NOs: 61, 62, 63 and 64 over a contiguous stretch of about 20 amino acids up to the full length of any one of SEQ ID NOs: 61, 62, 63 and 64.

The peptide mimetic can comprise an N-terminal domain as described herein, for example, when the amino acid sequence the peptide mimetic starting from the N-terminus (or in other words, starting from the first amino acid of the peptide mimetic) corresponds to that of the specific N-terminal domain described herein (e.g, SEQ ID NOs: 59, 62, 63, or 64). For example, when amino acids 1 to 21 of the peptide mimetic are SEQ ID NO: 59, 62, 63, or 64.

The C-terminal domain may also be generalized in a manner like that of the N-terminal domain. The C-terminal 152 amino acid residues of cpDKK3b (e.g., amino acids 174 to 326 of SEQ ID NO: 2) is replaced with about 12 to about 14 random amino acids so long as the sequence forms a random coil, alpha helix, or β-pleated sheet and includes at least two or three negatively charged amino acids within the last 6 amino acids of the C-terminal domain.

This strategy may be further optimized by including in the last 6 amino acids at least two consecutive, negatively charged amino acid residues or at least two consecutive amino acid residues wherein one is a negatively charged amino acid and wherein one is a positively charged amino acid. Preferably, the consecutive, charged amino acids are positioned just prior to the last amino acid of the C-terminal domain. For example, if the amino acid position of the last amino acid of the C-terminus is w then the two consecutive charged amino acids that are “just prior” to the last amino acid are at positions ψ-1 and ψ-2. Similarly, if the sequence includes three consecutive charged amino acids “just prior” to the last amino acid, then the three consecutive charged amino acids are positioned at positions ψ-1, ψ-2, and ψ-3. A preferred C-terminal domain comprises or consists of the following amino acid sequence: TAALLIILGGDDI (SEQ ID NO: 60). Another example of a C-terminal domain comprises or consists of the following amino acid: TSQLLIILGGDDI (SEQ ID NO: 49).

The peptide mimetic can comprise a C-terminal domain as described herein, for example, when the amino acid sequence at the C-terminus of the peptide mimetic (ending at the last amino acid) is the sequence of the specific C-terminal domain as described herein (e.g, SEQ ID NOs: 60); or in other words, the last 13 amino acids of the peptide mimetic are SEQ ID NO: 60.

Variants of SEQ ID NO: 60 or SEQ ID NO: 49 with one or more amino acid additions, deletions, and/or conservative substitutions that preserve the inhibitory activity on β-catenin nuclear translocation are also contemplated. The effects of the amino acid addition(s), deletion(s), and/or substitution(s) on the activity of any variant can be tested using routine methods and assays known in the art and described in the examples. Preferably, peptide mimetics of the invention can include an C-terminal domain having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity or similarity to the sequences of SEQ ID NO: 60. Preferably, peptide mimetics of the invention can include an N-terminal domain having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity or similarity to the sequences of SEQ ID NO: 60 over a contiguous stretch of about 20 amino acids up to the full length of SEQ ID NO: 60. In additional aspects, peptide mimetics of the invention can include an C-terminal domain having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity or similarity to the sequences of SEQ ID NO: 49. In further aspects, peptide mimetics of the invention can include an C-terminal domain having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity or similarity to the sequences of SEQ ID NO: 49 over a contiguous stretch of about 20 amino acids up to the full length of SEQ ID NO: 49.

With regard to the above described generalization of the N- and C-termini of the peptide mimetics of the invention, and without being limited to any one scientific theory, deletion analysis revealed that residues 10-70 of human wild type DKK3b (FIG. 1, SEQ ID NO: 47) can be eliminated without inactivating the protein. The sequence length of 20-21 residues was selected because a proline dimer was positioned 20 residues from the N-terminus in human wild type human DKK3b (SEQ ID NO: 47) and will introduce a “kink” in the protein that may be critical. The loss of one or more the negative charged residues at positions 2, 4 or 5 of the human wild type DKK3b inactivates the protein, and therefore it appears that the negative charge at those positions appears to be essential.

The N-1 domain of DKK3b is the critical domain required for silencing of β-catenin signaling. Alignment of the N-1 domains of all human DKK family members revealed considerable organizational conservation raising the possibility that this domain in all family members may function like that of human wild type DKK3b. Experiments evaluating the impact of exchanging the N-1 domain of DKK3b for that of another family member indicated that this was possible while retaining inhibition of β-catenin by modified protein. The N-1 domain of the peptide mimetic described herein can be selected, for example, from the N-1 domain of native human DKK1, native human DKK2, native human DKK3b and native human DKK4, or can be a variant of the N-1 domain of native human DKK1, native human DKK2, native human DKK3b and native human DKK4. The amino acid sequences for the respective N-1 domains of each of the human DKK family members is as follows:

DKKI RESIDUES 74-141 (GenBank: AAQ89364)
(SEQ ID NO: 3)
QTIDNYQPYPCAEDEECGTDEYCASPTRGGDAGVQICLACRKRRKRCMRH
AMCCPGNYCKNGICVSS;
DKK2 RESIDUES 78-130 (GenBank: AAQ88780)-
(SEQ ID NO: 4)
CSSDKECEVGRYCHSPHQGSSACMVCRRKKKRCHRDGMCCPSTRCNNGIC
IPV;
DKK3b RESIDUES 74-126
(SEQ ID NO: 5)
HECIIDEDCGPSMYCQFASFQYTCQPCRGQRMLCTRDSECCGDQLCVWGH
CTK;
and
DKK4 RESIDUES 31-91 (GenBank: NP 055235)
(SEQ ID NO: 6)
DLHGARKGSQCLSDTDCNTRKFCLQPRDEKPFCATCRGLRRRCQRDAMCC
PGTLCVNDVCT.

Variants of these sequences with one or more amino acid additions, deletions, and/or conservative substitutions that preserve the inhibitory activity on β-catenin nuclear translocation are also contemplated. The effects of the amino acid addition(s), deletion(s), and/or substitution(s) on the activity of any variant can be tested using routine methods and assays known in the art and described in the examples. Preferably peptide mimetics of the invention can include an N-1 domain having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity or similarity to the sequences of SEQ ID NOs: 3, 4, 5 and 6. Preferably, the respective variants of SEQ ID NOS: 3, 4, 5 and 6 have an amino acid sequence having sequence identity that is about 75%, 80%, 85%, 90%, 91%, 92%, 93%, 94%, 95%, 96%, 97%, 98%, 99% or higher over a contiguous stretch of about 20 amino acids up to the full length of SEQ ID NOS: 3, 4, 5 and 6.

An important feature of the N-1 domain of the engineered peptide mimetic is the inclusion of a cell penetrating peptide within the N-1 domain instead of fusing an exogenous cell penetrating peptide to, for example, the N-terminal domain of the peptide mimetic. Including the peptide domain “internally” within the peptide mimetic reduces the antigenicity of the peptide mimetic.

The cell penetrating peptide (cp) comprises an amino acid sequence that is known to have cell penetrating capabilities. The terms “cell-penetrating domain,” “cell-penetrating region,” and “cell-penetrating peptide” are used interchangeably herein. Cell-penetrating peptides are short (typically about 4-40 amino acids) peptides that are able to cross cell membranes. Peptides referred to as “nuclear localization sequences” are a subset of cell penetrating peptides. Cell penetrating peptides are typically water soluble, cationic or amphipathic, and rich in basic amino acids (e.g., lysine and/or arginine residues). Cell penetrating peptides can also be positively charged amphipathic peptides, or peptides that are hydrophobic, containing only apolar residues with low net charge or hydrophobic amino acid groups.

Preferably cell penetrating amino acid sequences are those found in the human wild type DKK family members that are secreted proteins. The native/intrinsic cell penetrating domains of the individual human DKK family members are listed in Table 2.

TABLE 2

| NAME | AMINO ACID SEQUENCE | SEQ ID NO |
|---|---|---|
| DKK1 | CLACRKRRKRCMRHMCC | (SEQ ID NO: 54) |
| DKK2 | CMVCRRKKKRCHRDGMCC | (SEQ ID NO: 55) |
| DKK3B | CQPCRGQRMLCTRDSECC | (SEQ ID NO: 56) |
| DKK4 | CATCRGLRRRCQRDAMCC | (SEQ ID NO: 57) |

Other Representative but non-limiting cell penetrating amino acid sequences are shown in Table 3.

TABLE 3

| NAME | AMINO ACID SEQUENCE | SEQ ID NO |
|---|---|---|
| C. elegans | FKKFRKF | (SEQ ID NO: 9) |
| SDC3 | | |
| CADY-K | GLWRALWRLLRSLWRLLWK | (SEQ ID NO: 10) |
| EB1 CPP | LIRLWSHLIHIWFQNRRLK | (SEQ ID NO: 11) |
| | WKKK | |
| FBP CPP | GALFLGWLGAAGSTMGAWS | (SEQ ID NO: 12) |
| | QPKKKRKV | |
| FGF4 CPP | AAVALLPAVLLALLAP | (SEQ ID NO: 13) |
| HATF3 | ERKKRRRE | (SEQ ID NO: 14) |
| hCT CPP | LGTYTQDFNKTFPQTAIGV | (SEQ ID NO: 15) |
| | GAP | |
| MAP CPP | KLALKLALKALKAALKLA | (SEQ ID NO: 16) |
| MPG CPP | GLAFLGFLGAAGSTMGAWS | (SEQ ID NO: 17) |
| | QPKKKRKV | |
| NF-κB | VQRKRQKLMP | (SEQ ID NO: 18) |
| OCT-6 | GRKRKKRT | (SEQ ID NO: 19) |
| Penetratin | RQIKIWFQNRRMKWKK | (SEQ ID NO: 20) |
| CPP | | |
| Penetratin | RQLKLWFQNRRMKWKK | (SEQ ID NO: 21) |
| CPP | | |
| variant 1 | | |
| Penetratin | REIKIWFQNRRMKWKK | (SEQ ID NO: 22) |
| CPP | | |
| variant 2 | | |
| Pep-1 CPP | KETWWETWWTEWSQPKKRK | (SEQ ID NO: 23) |
| | V | |
| plsl CPP | PVIRVWFQNKRCKDKK | (SEQ ID NO: 24) |
| Poly-Arg | RRRR(R)₀₋₆ | (SEQ ID NO: 25) |
| CPP | | |
| pVEC CPP | LLIILRRRIRKQAHAH | (SEQ ID NO: 26) |
| RL-16 CPP | RRLRRLLRRLLRRLRR | (SEQ ID NO: 27) |
| RVGCPP | RVGRRRRRRRRR | (SEQ ID NO: 28) |
| ReW3 CPP | RRWWRRWRR | (SEQ ID NO: 29) |
| SBP CPP | MGLGLHLLVLAAALQGAWS | (SEQ ID NO: 30) |
| | QPKKKRKV | |
| SV40 | PKKKRKV | (SEQ ID NO: 31) |
| SynB1 CPP | RGGRLSYSRRFSTSTGR | (SEQ ID NO: 32) |
| SynB3 CPP | RRLSYSRRRF | (SEQ ID NO: 33) |
| SynB5 CPP | RGGRLAYLRRRWAVLGR | (SEQ ID NO: 34) |
| Tat⁴⁷⁻⁵⁷ CPP | YGRKKRRQRRR | (SEQ ID NO: 35) |
| Tat⁴⁷⁻⁵⁶ CPP | YGRKKRRQRR | (SEQ ID NO: 36) |
| Tat⁴⁸⁻⁵⁶ CPP | GRKKRRQRR | (SEQ ID NO: 37) |
| Tat⁴⁸⁻⁶⁰ CPP | GRKKRRQRRRPPQ | (SEQ ID NO: 38) |
| TCF1-α | GKKKKRKREKL | (SEQ ID NO: 39) |
| TFIIE-β | SKKKKTKV | (SEQ ID NO: 40) |
| TP CPP | GWTLNSAGYLLGKINLKAL | (SEQ ID NO: 41) |
| | AALAKKIL | |
| TP10 CPP | AGYLLGKINLKALAALAKK | (SEQ ID NO: 42) |
| | IL | |
| TP2 CPP | PLIYLRLLRGQF | (SEQ ID NO: 43) |
| VP22 CPP | DAATATRGRSAASRPTQR | (SEQ ID NO: 44) |
| | PRAPARSASRPRRPVQ | |
| Variant | WLRRIKAWLRRIKA | (SEQ ID NO: 51) |
| Variant | YGRKKRRQRRR | (SEQ ID NO: 52) |
| Variant | YARAAARQARA | (SEQ ID NO: 53) |
| Poly-Arg | RRRRRR(R)₁₋₆ | (SEQ ID NO: 81) |
| CPP | | |

Because the function of a cell penetrating peptide depends on their physical characteristics rather than sequence-specific interactions, the cell penetrating peptide can have the reverse sequence of those provided in Table 2 and/or known in the art; thus, for example, the reverse sequence of SEQ ID NO: 53 is ARAQRAAARAY (reading amino acids from left to right as N-terminal side to the C-terminal side) which can be used a cell penetrating peptide. Variants of these sequences with one or more amino acid additions, deletions, and/or conservative substitutions that retain the ability to cross cell membranes and are also suitable for use in the invention. The effect of the amino acid addition(s), deletion(s), and/or substitution(s) on the ability of the CPP to mediate cell penetration can be tested using routine methods known in the art.

One preferred cell penetrating peptide is a poly-arginine peptide comprising from about 4 to about 8 amino acids and preferably about 6 amino acids; including, for example, SEQ ID NO: 25. Preferably, the cell penetrating peptide is located in the N-1 domain of the DKK protein family. Preferably the cell penetrating peptide is located in Loop 2 of the N-1 domain. Loop 2 of the N-1 domain occurs around amino acids 100 to 114 of SEQ ID NO: 47, human wild type DKK3b. By analogy, Loop 2 of the N-1 domain occurs around amino acids 99 to 117 of human wild type DKK2 (Gen Bank Acc. No. AAQ88780.1).

Another preferred feature of the peptide mimetic is that the N-1 domain of the peptide mimetic is cysteine deficient. As used herein, the N-1 domain “cysteine deficient” when at least one of the cysteine residues in the wild type, or naturally occurring N-1 domain is replaced with a conservative amino acid. Preferably, one or more cysteine residues is replaced preferably with alanine, threonine or serine; in yet other preferred aspects one or more cysteine residues is replaced with alanine or serine. For example, one or more cysteine residues of the N-1 domain of human DKK1, the N-1 domain of human DKK2, the N-1 domain of human DKK3b, or the N-1 domain of human DKK4 is replaced with alanine or serine. Preferably at least 3, 4, 5, 6, 7, 8, 9 or 10 the cysteine residues present in the N-1 domain are substituted, preferably with alanine or serine. Replacement of one or more cysteine residues reduces or eliminates aggregation of the protein mimetic during recombinant production due to disulfide bridging. Aggregation has been found to be a confounding problem routinely observed during recombinant production of DKK3b proteins and variants thereof in both prokaryotic and eukaryotic protein expression systems.

Thus, the N-1 domain of the peptide mimetic can be a variant of one of the N-1 domains of human DKK1, human DKK2, human DKK3b, and human DKK4 (for example, one of SEQ ID NOs. 3, 4, 5 and 6), wherein a cell penetrating peptide is added to the N-1 domain of human DKK1, human DKK2, human DKK3b, and human DKK4. In preferred aspects, the cell penetrating domain is SEQ ID NO: 25; optionally, wherein the cell penetrating domain is six consecutive arginine residues (RRRRRR). For example, the N-1 domain of human DKK2 is SEQ ID NO: 4:

CSSDKECEVGRYCHSPHQGSSACMVCRRKKKRCHRDGMCCPSTRCNNGIC
IPV.

In certain aspects, the N-1 domain of the peptide mimetic is a variant of SEQ ID NO: 4 wherein the cell penetrating domain, RRRRRR, is added to Loop 2. For example, an exemplary variant of SEQ ID NO: 4 comprising the cell penetrating peptide in Loop 2 is: CSSDKECEVGRYCHSPHQGSSACMVCRRRRRRCHRDGMCCPSTRCNNGICIPV (SEQ ID NO: 73). In SEQ ID NO: 73, the underlined portion of SEQ ID NO: 4 (shown above) is replaced with RRRRRR.

In yet additional aspects, the peptide mimetic is a variant of one of the N-1 domains of human DKK1, human DKK2, human DKK3b, and human DKK4 (for example, one of SEQ ID NOs. 3, 4, 5 and 6), wherein one or more cysteine residues in the N-1 domain (of human DKK1, human DKK2, human DKK3b, and human DKK4) is replaced with a conservative amino acid, for example, serine or alanine. In yet further aspects, the peptide mimetic is a variant of one of the N-1 domains of human DKK1, human DKK2, human DKK3b, and human DKK4 (for example, one of SEQ ID NOs. 3, 4, 5 and 6), wherein a cell penetrating peptide is added to the N-1 domain of human DKK1, human DKK2, human DKK3b, and human DKK4, and wherein one or more cysteine residues in the N-1 domain (of human DKK1, human DKK2, human DKK3b, and human DKK4) is replaced with a conservative amino acid, for example, serine or alanine. In preferred aspects, the cell penetrating domain is SEQ ID NO: 25; optionally, wherein the cell penetrating domain is six consecutive arginine residues (RRRRRR). For example, one or more of the underlined cysteine residue shown in SEQ ID NO: 73 are replaced with serine or alanine:

(SEQ ID NO: 73)
CSSDKECEVGRYCHSPHQGSSACMVCRRRRRRCHRDGMCCPSTRCNNGIC
IPV.

A specific example of N-1 domain that is a variant of SEQ ID NO: 4 is: ASSDKESEVGRYSHSPHQGSSACMVARRRRRRAHRDGMACPSTRSNNGIAIPV (SEQ ID NO: 69), wherein the underlined residues are alanine or serine residues that replaced cysteine residues in SEQ ID NO: 73. Another example of an N-1 domain that is a variant of SEQ ID NO: 4 is: ASSDKESEVGRYSHSPHQGSSAωMVARRRRRRAHRDGMAωPSTRSNNGIAIPV (SEQ ID NO: 74), wherein each ω is independently alanine or serine and wherein the underlined residues are alanine or serine residues that replaced cysteine residues in SEQ ID NO: 73.

Preferred N-1 domains of the peptide mimetic that have been modified to be cysteine deficient and include a cell penetrating peptide include the following:

DKK3b -
(SEQ ID NO: 7)
HEAIIDEDSGPSMYSQFASFQYTCQPARRRRRRATRDSESCGDQLAVWGH
STK;
DKKI -
(SEQ ID NO: 8)
QTIDNYQPYPAAEDEESGTDEYSASPTRGGDAGVQICLAARRRRRRAMRH
AMSCPGNYAKNGIAVSS;
DKK2 -
(SEQ ID NO: 45)
KGKNLGQAYPASSDKESEVGRYSHSPHQGSSACMVARRRRRRAHRDGMAC
PSTRSNNGIAIPV;
and
DKK4 -
(SEQ ID NO: 46)
DLHGARKGSQALSDTDSNTRKFSLQPRDEKPFCATARRRRRRAQRDAMSC
PGTLSVNDVAT.

In additional aspects, a preferred N-1 domain of the peptide mimetic that is cysteine deficient and includes a cell penetrating domain comprises or consists of:

(SEQ ID NO: 69)
ASSDKESEVGRYSHSPHQGSSACMVARRRRRRAHRDGMACPSTRSNNGIA
IPV.

In some embodiments, the N-1 domain of the peptide mimetic is a variant of an N-1 domain from human DKK1, human DKK2, human DKK3b or human DKK4, for example, a variant of any one of SEQ ID NOs: 3, 4, 5 and 6. For example, the variant can comprise a cell penetrating peptide. In additional examples, the variant can comprise one or more amino acid substitutions, wherein one or more cysteine residues of the N-1 domain of human DKK1, human DKK2, human DKK3b or human DKK4 is replaced/substituted with another amino acid, for example, a conservative amino acid, including for example, substitution with alanine or serine.

In certain aspects, the N-1 domain of the peptide mimetic is a variant of an N-1 domain from human DKK1, human DKK2, human DKK3b or human DKK4, for example, a variant of any one of SEQ ID NOs: 3, 4, 5 and 6, wherein:
i) the variant comprises a cell penetrating peptide; and optionally wherein at least one least one cysteine of the N-1 domain of human DKK1, human DKK2, human DKK3b or human DKK4 is replaced/substituted with another amino acid, for example, a conservative amino acid, including for example, substitution with alanine or serine; and
ii) optionally, the variant has at least about 75%, at least about 76%, at least about 77%, at least about 78%, at least about 79%, or at least about 80% sequence identity to an N-1 domain from human DKK1, human DKK2, human DKK3b or human DKK4, or at least about 75%, at least about 76%, at least about 77%, at least about 78%, at least about 79%, or at least about 80% sequence identity to one of SEQ ID NOs: 3, 4, 5 and 6.

The variant comprises a cell penetrating peptide, for example, when the cell penetrating peptide is added to or incorporated within the amino acid sequence of the N-1 domain of human DKK1, human DKK2, human DKK3b or human DKK4; for example, the cell penetrating peptide can be located at the N-terminal end or the C-terminal end, or the cell penetrating peptide can be located within the N-1 domain, for example, within Loop 2 as described in more detail below. The variant can further comprise substitution of at least one cysteine of the N-1 domain of human DKK1, human DKK2, human DKK3b or human DKK4 with a conservative amino acid. In certain aspects, the variant has at least about 75%, at least about 76%, at least about 77%, at least about 78%, at least about 79%, or at least about 80% sequence identity, at least about 85%, at least about 90%, at least about 95%, at least about 97%, at least about 98%, or at least about 99% sequence identity to one of SEQ ID NOs: 3, 4, 5, and 6.

In certain aspects, the N-1 domain of the peptide mimetic is a variant of an N-1 domain from human DKK1, human DKK2, human DKK3b or human DKK4, wherein:
i) the variant comprises a cell-penetrating peptide, and optionally wherein at least one least one cysteine of the N-1 domain of human DKK1, human DKK2, human DKK3b or human DKK4 is replaced/substituted with another amino acid, for example, a conservative amino acid, including for example, substitution with alanine or serine; and
ii) optionally, has at least about 80% sequence identity to one of SEQ ID NOs: 7, 8, 45, 46 or 69.

In yet additional aspects, the N-1 domain of the peptide mimetic has at least about 85%, at least about 90%, at least about 95%, at least about 98%, or at least about 99% sequence identity to the amino acid sequence of SEQ ID NOs: 7, 8, 45, 46, or 69 and wherein the N-1 domain further comprises a cell-penetrating peptide. In certain preferred aspects, the N-1 domain has at least about 80%, at least about 85%, at least about 90%, at least about 95%, at least about 97%, at least about 98%, or at least about 99% sequence identity to one of SEQ ID NO: 45. In further aspects, the N-1 domain has at least about 80%, at least about 85%, at least about 90%, at least about 95%, at least about 97%, at least about 98%, or at least about 99% sequence identity to one of SEQ ID NO: 69.

In certain aspects, the cell penetrating peptide is located within Loop 2 of the N-1 domain described in more detail above.

Variants of these sequences with one or more amino acid additions, deletions, and/or conservative substitutions that preserve the preserves the inhibitory activity on β-catenin nuclear translocation are also contemplated. The effects of the amino acid addition(s), deletion(s), and/or substitution(s) on the activity of any variant can be tested using routine methods and assays known in the art and described in the examples. Preferably peptide mimetics of the invention can include an N-1 domain having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the sequences of SEQ ID NOS: 7, 8, 45 and 46.

Preferably, a peptide mimetic of the invention comprises an N-terminal domain having the amino acid sequence of SEQ ID NO: 59. Preferably a peptide mimetic of the invention comprises a C-terminal domain having the amino acid sequence of SEQ ID NO: 60. Preferably a peptide mimetic of the invention comprises or consists of an N-1 domain having the amino acid sequence of SEQ ID NO: 45. A preferred peptide mimetic is referred to herein as “AC1” and comprises or consists of the amino acid sequence of SEQ ID NO: 1 as follows: MDAEDLLLKLNLAATVGTAPPKGKNLGQAYPASSDKESEVGRYSHSPHQGSSACMVA RRRRRRAHRDGMACPSTRSNNGIAIPVPTAALLIILGGDDI (SEQ ID NO: 1). In yet further aspects, the peptide mimetic has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to SEQ ID NO: 1. An additional preferred peptide mimetic comprises or consists of the amino acid sequence of SEQ ID NO: 1 wherein the starting methionine is replaced with serine, threonine, or a non-polar amino acid other than proline and methionine. For example, the preferred peptide mimetic can comprise SEQ ID NO: 65 as follows: DAEDLLLKLNLAATVGTAPPKGKNLGQAYPASSDKESEVGRYSHSPHQGSSACMVARR RRRRAHRDGMACPSTRSNNGIAIPVPTAALLIILGGDDI (SEQ ID NO: 65) (starting at amino acid 2). In yet further aspects, the preferred peptide mimetic comprises or consists of the following sequence: Φ₂DAEDLLLKLNLAATVGTAPPKGKNLGQAYPASSDKESEVGRYSHSPHQGSSACMVA RRRRRRAHRDGMACPSTRSNNGIAIPVPTAALLIILGGDDI (SEQ ID NO: 66), wherein Φ₂ is a nonpolar amino acid other than proline (including, for example, methionine, alanine, and isoleucine), or is serine or threonine; in certain aspects, Φ₂ is threonine, serine or a nonpolar amino acid other than proline and methionine. In yet further aspects, the peptide mimetic comprises or consists of one of the following sequences: ADAEDLLLKLNLAATVGTAPPKGKNLGQAYPASSDKESEVGRYSHSPHQGSSACMVAR RRRRRAHRDGMACPSTRSNNGIAIPVPTAALLIILGGDDI (SEQ ID NO: 67) or IDAEDLLLKLNLAATVGTAPPKGKNLGQAYPAS SDKESEVGRYSHSPHQGS SACMVAR RRRRRAHRDGMACPSTRSNNGIAIPVPTAALLIILGGDDI (SEQ ID NO: 68). Preferably a peptide mimetic of the invention comprises an N-1 domain having the amino acid sequence of SEQ ID NO: 45). In yet further aspects, the peptide mimetic has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to SEQ ID NO: 66, 67, or 68.

In additional preferred embodiments, the peptide mimetic comprises or consists of: Φ₂DAEDLLLKLNLAATVGTAPPKGKNLGQAYPASSDKESEVGRYSHSPHQGSSAωMVAR RRRRRAHRDGMAωPSTRSNNGIAIPVPTAALLIILGGDDI (SEQ ID NO: 70), wherein Φ₂ is as defined above and wherein each co is independently serine or alanine. In yet further aspects, the peptide mimetic comprises or consists of one of the following sequences: ADAEDLLLKLNLAATVGTAPPKGKNLGQAYPASSDKESEVGRYSHSPHQGSSAωMVAR RRRRRAHRDGMAωPSTRSNNGIAIPVPTAALLIILGGDDI (SEQ ID NO: 71) or IDAEDLLLKLNLAATVGTAPPKGKNLGQAYPASSDKESEVGRYSHSPHQGSSAωMVAR RRRRRAHRDGMAωPSTRSNNGIAIPVPTAALLIILGGDDI (SEQ ID NO: 72). In yet further aspects, the peptide mimetic has a sequence selected from the group consisting of:

(SEQ ID NO: 75)
IDAEDLLLKLNLAATVGTAPPKGKNLGQAYPASSDKESEVGRYSHSPHQG
SSASMVARRRRRRAHRDGMAAPSTRSNNGIAIPVPTAALLIILGGDDI;
(SEQ ID NO: 76)
IDAEDLLLKLNLAATVGTAPPKGKNLGQAYPASSDKESEVGRYSHSPHQG
SSAAMVARRRRRRAHRDGMASPSTRSNNGIAIPVPTAALLIILGGDDI;
(SEQ ID NO: 77)
ADAEDLLLKLNLAATVGTAPPKGKNLGQAYPASSDKESEVGRYSHSPHQG
SSASMVARRRRRRAHRDGMAAPSTRSNNGIAIPVPTAALLIILGGDDI;
(SEQ ID NO: 78)
ADAEDLLLKLNLAATVGTAPPKGKNLGQAYPASSDKESEVGRYSHSPHQG
SSAAMVARRRRRRAHRDGMASPSTRSNNGIAIPVPTAALLIILGGDDI;
(SEQ ID NO: 79)
MDAEDLLLKLNLAATVGTAPPKGKNLGQAYPASSDKESEVGRYSHSPHQG
SSASMVARRRRRRAHRDGMAAPSTRSNNGIAIPVPTAALLIILGGDDI;
or
(SEQ ID NO: 80)
MDAEDLLLKLNLAATVGTAPPKGKNLGQAYPASSDKESEVGRYSHSPHQG
SSAAMVARRRRRRAHRDGMASPSTRSNNGIAIPVPTAALLIILGGDDI.

In yet another aspect, a peptide mimetic of the invention comprises an N-terminal domain having the amino acid sequence of SEQ ID NO: 48. Preferably a peptide mimetic of the invention comprises a C-terminal domain having the amino acid sequence of SEQ ID NO: 49. Preferably a peptide mimetic of the invention comprises an N-1 domain having the amino acid sequence of SEQ ID NO: 45.

Variants of SEQ ID NO: 1 with one or more amino acid additions, deletions, and/or conservative substitutions that preserve the inhibitory activity on β-catenin nuclear translocation are also contemplated. The effects of the amino acid addition(s), deletion(s), and/or substitution(s) on the activity of any variant can be tested using routine methods and assays known in the art and described in the Examples. Preferably peptide mimetics of the invention comprise an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity or similarity to SEQ ID NO: 1. Preferably the identity or similarity is calculated over a defined length of contiguous amino acids (e.g., a “comparison window”). For example, a comparison window may be a contiguous stretch of about 20 amino acids up to the full length of SEQ ID NO: 1.

Variants of SEQ ID NOs: 65, 66, 67, 68, 69, 70, 71 and 72 with one or more amino acid additions, deletions, and/or conservative substitutions that preserve the preserves the inhibitory activity on β-catenin nuclear translocation are also contemplated. The effects of the amino acid addition(s), deletion(s), and/or substitution(s) on the activity of any variant can be tested using routine methods and assays known in the art and described in the Examples. Preferably peptide mimetics of the invention comprise an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity or similarity to SEQ ID NOs: 65, 66, 67 and 68. Preferably the identity or similarity is calculated over a defined length of contiguous amino acids (e.g., a “comparison window”). For example, a comparison window may be a contiguous stretch of about 20 amino acids up to the full length of SEQ ID NOs: 65, 66, 67, 68, 69, 70, 71 and 72.

In certain aspects, the peptide mimetic comprises:
i) an N-terminal domain that has at least about 80%, at least about 85%, at least about 90%, at least about 95%, at least about 98%, or at least about 99% sequence identity to SEQ ID NOs: 59, 62, 63, and 64;
ii) an N-1 domain that has at least about 80%, at least about 85%, at least about 90%, at least about 95%, at least about 98%, or at least about 99% sequence identity to SEQ ID NO: 7, 8, 45, 46, or 69; preferably, at least about 80%, at least about 85%, at least about 90%, at least about 95%, at least about 98%, or at least about 99% sequence identity to SEQ ID NO: 45 or 69; and
iii) a C-terminal domain that has at least about 80%, at least about 85%, at least about 90%, at least about 95%, at least about 98%, or at least about 99% sequence identity to SEQ ID NO: 60.

The peptide mimetic can also comprise an amino acid linker between one or more of the N-terminal domain, N-1 domain and C-terminal domain. The peptide mimetic can, for example, comprise an amino acid linker between the N-terminal domain and the N-1 domain, and/or an amino acid linker between the N-1 domain and the C-terminal domain. Preferably, the amino acid linkers is about 1 to about 150 amino acids in length. In certain aspects, the peptide mimetic comprises an amino acid linker between the N-terminal domain and the N-1 domain, wherein the amino acid linker is between about 1 and about 70 amino acids in length; or about 1 to about 50 amino acids in length; or about 1 to about 30 amino acids in length; or about 1 to about 20 amino acids in length. In yet other aspects, the amino acid linker between the N-terminal domain and the N-1 domain is about 1 or 2 amino acids in length. In certain additional aspects, the peptide mimetic comprises an amino acid linker between the N-1 domain and the C-terminal domain, wherein the amino acid linker is between about 1 to about 150 amino acids in length; or about 1 to about 125 amino acids in length; or about 1 to about 100 amino acids in length; or about 1 to about 75 amino acids in length; or about 1 to about 50 amino acids in length; or about 1 to about 30 amino acid in length; or about 1 to about 20 amino acids in length. In certain aspects, the amino acid linker between the N-1 domain and the C-terminal domain is about 1 to about 2 amino acids in length.

The peptide mimetic of the invention may also be characterized as follows. A peptide mimetic of DKK3b capable of inhibiting β-catenin nuclear translocation or capable of inhibiting a β-catenin signaling pathway comprising from N-terminus to C-terminus:
i) an N-terminal domain of comprising a peptide of Formula 1:

NH₂-Met-Y-X-Y-Y-X₁ Formula 1

wherein:
Y is a negatively charged amino acid;
X is any non-polar amino acid or negatively charged amino acid;
X₁ is a peptide of about 4 to about 70 amino acids, about 4 to about 20 amino acid, or about 4 to about 15 amino acids, that forms a random coil, α-helix or β-pleated sheet; and
Met is the amino acid, methionine;
ii) an N-1 domain comprising an amino acid sequence that is at least about 80% identical to the N-1 domain of DKK1, DKK2, DKK3b, or DKK4 wherein the N1 domain further comprises a cell penetrating domain; and
iii) a C-terminal domain comprising the peptide of Formula 2

X₂-Gly-Gly-X₃-Ile-COOH (Formula 2)

wherein:
X₂ is a peptide of about 4 to about 40, about 4 to about 20 amino acid, about 4 to about 15 amino acids, or about 4 to about 8 amino acids that form a random coil, α-helix, or β-pleated sheet; Gly is the amino acid, glycine; and
X₃ is a peptide of 2 consecutive amino acids in length comprising 2 negatively charged amino acids or one negatively charged amino acid and one positively charged amino acid;
preferably wherein Y is glutamic acid (Glu) or aspartic acid (Asp); preferably wherein Xi is about 15-17 amino acids in length; preferably wherein the N-terminal domain is about 19 to about 22 amino acids in length; preferably X is alanine; preferably wherein the N-terminal domain comprises a peptide of Formula 3:

NH₂-Met-Glu-X₄-Asp-Glu-X₁ Formula 3a; or

NH₂-Met-Asp-X₄-Glu-Asp-X₁ Formula 3b

wherein X₄ is any hydrophobic amino acid; and X₁ is a peptide of about 4 to about 70 amino acids that forms a random coil, α-helix or β-pleated sheet; preferably wherein X₄ of Formula 3a or Formula 3b is alanine; preferably wherein X₁ of Formula 3a or Formula 3b is 15-17 amino acids in length; preferably wherein the peptide of Formula 3a or Formula 3b has the amino acid sequence of SEQ ID NO: 59; or alternatively, wherein the peptide of Formula 3a or Formula 3b has the amino acid sequence of SEQ ID NO: 48; preferably wherein the N-1 domain of DKK1, DKK2, DKK3b, or DKK4 is modified to substitute at least one of the cysteine residues present in the native N-1 domain of DKK1, DKK2, DKK3b, or DKK4 with a conservative amino acid substitution; preferably the conservative amino acid substitution is selected from alanine (Ala) and serine (Ser); preferably wherein the cell penetrating peptide is about 4 to about 8 amino acids in length; preferably wherein the cell penetrating peptide is about 6 amino acids in length; preferably wherein the cell penetrating peptide comprises or consists of 6 arginine residues; preferably wherein N-1 comprises the N-1 domain of DKK2; preferably wherein the N-1 domain of DKK2 is modified to substitute at least 1 of the cysteine residues with a conservative amino acid substitution; preferably wherein at least 8 of the cysteine residues of the N-1 domain of DKK2 are substituted with a conservative amino acid substitution; preferably wherein all of the cysteine residues of the N-1 domain are substituted with a conservative amino acid substitution; preferably wherein the conservative amino acid substitution is selected from alanine (Ala) and serine (Ser); preferably wherein the amino acid sequence of the N-1 domain is selected from SEQ ID NOS: 7, 8, 45, 46, and 69, preferably wherein X2 is about 8 amino acids in length; preferably wherein the C-terminal domain comprises about 12 to about 14 amino acids; preferably wherein X3 comprises at least two consecutive, negatively charged amino acids wherein each negatively charged amino acid is independently selected from Asp and Glu; or alternatively wherein X3 comprises at least two consecutive charged amino acids wherein one amino acid residue is a positively charged and is selected from Lysine (Lys) and Arginine (Arg) and wherein one amino acid residue negatively charged and is selected from aspartate (Asp) and glutamic acid (Glu); preferably, the C-terminal domain has the amino acid sequence of SEQ ID NO: 60; or alternatively, wherein the C-terminal domain of Formula 2 has the amino acid sequence of SEQ ID NO: 49.

In further embodiments, the peptide mimetic of the invention can also be characterized as follows. A peptide mimetic of DKK3b capable of inhibiting β-catenin nuclear translocation or capable of inhibiting a β-catenin signaling pathway comprising from N-terminus to C-terminus:
i) an N-terminal domain of comprising a peptide of Formula 4

NH₂-Φ-Y-X-Y-Y-X₁ Formula 4

wherein:
each Y is independently a negatively charged amino acid;
X is any non-polar amino acid or negatively charged amino acid;
X₁ is a peptide of about 4 to about 70, about 4 to about 20 amino acid, or about 4 to about 15 amino acids that forms a random coil, α-helix or β-pleated sheet; and
Φ is a nonpolar amino acid other than proline, or is threonine or serine; in some embodiments, Φ is methionine, alanine, isoleucine, serine, or threonine.
ii) an N-1 domain that is a variant of the N-1 domain of human DKK1 having the amino acid sequence of SEQ ID NO: 3, a variant of the N-1 domain of DKK2 having the amino acid sequence of SEQ ID NO: 4, a variant of the N-1 domain of DKK3b having the amino acid sequence of SEQ ID NO: 5, or a variant of the N-1 domain of DKK4 having the amino acid sequence of SEQ ID NO: 6; wherein the variant comprises a cell-penetrating peptide and wherein the variant has at least about 80% sequence identity to one of SEQ ID NOs: 3, 4, 5 and 6, or
an N-1 domain that has at least about 80% sequence identity to the amino acid sequence of SEQ ID NO: 7, 8, 45, 46 and 69, wherein the N-1 domain further comprises a cell-penetrating peptide; and
iii) a C-terminal domain comprising the peptide of Formula 2

X₂- Gly-Gly-X₃-Ile-COOH (Formula 2)

wherein:
X₂ is a peptide of about 4 to about 40, about 4 to about 20 amino acid, about 4 to about 15 amino acids, or about 4 to about 8 amino acids that form a random coil, α-helix, or β-pleated sheet;
Gly is the amino acid, glycine; and
X₃ is a peptide of 2 consecutive amino acids in length comprising 2 negatively charged amino acids or one negatively charged amino acid and one positively charged amino acid;
preferably wherein Y is glutamic acid (Glu) or aspartic acid (Asp); preferably wherein X₁ is about 15-17 amino acids in length; preferably wherein the N-terminal domain is about 19 to about 22 amino acids in length; preferably X is alanine; preferably, wherein Φ is methionine, alanine or isoleucine; preferably wherein the N-terminal domain comprises a peptide of Formula 3:

NH₂-Φ-Glu-X₄-Asp-Glu-X₁ Formula 3a; or

NH₂-Φ-Asp-X₄-Glu-Asp-X₁ Formula 3b

wherein X₄ is any hydrophobic amino acid; and X₁ is a peptide of about 4 to about 70 amino acids that forms a random coil, α-helix or β-pleated sheet; preferably wherein X₄ of Formula 3a or Formula 3b is alanine; preferably wherein X₁ of Formula 3a or Formula 3b is 15-17 amino acids in length; preferably wherein the peptide of Formula 3a or Formula 3b has the amino acid sequence of SEQ ID NO: 62, 63 or 64; preferably wherein the N-1 domain has at least about 80% sequence identity to SEQ ID NO: 45 and comprises a cell penetrating domain; or preferably wherein the N-1 domain of DKK1, DKK2, DKK3b, or DKK4 is modified to substitute at least one of the cysteine residues present in the native N-1 domain of DKK1, DKK2, DKK3b, or DKK4 with a conservative amino acid substitution; preferably the conservative amino acid substitution is selected from alanine (Ala) and serine (Ser); preferably wherein the cell penetrating peptide is about 4 to about 8 amino acids in length; preferably wherein the cell penetrating peptide is about 6 amino acids in length; preferably wherein the cell penetrating peptide comprises or consists of 6 arginine residues; preferably wherein the N-1 domain comprises the N-1 domain of DKK2; preferably wherein the N-1 domain of DKK2 is modified to substitute at least one of the cysteine residues with a conservative amino acid substitution; preferably wherein at least 8 of the cysteine residues of the N-1 domain of DKK2 are substituted with a conservative amino acid substitution; preferably wherein all of the cysteine residues of the N-1 domain are substituted with a conservative amino acid substitution; preferably wherein the conservative amino acid substitution is selected from alanine (Ala) and serine (Ser); preferably wherein the amino acid sequence of the N-1 domain comprises or consists of one of SEQ ID NOs: 7, 8, 45, 46 and 69; preferably wherein X2 is about 8 amino acids in length; preferably wherein the C-terminal domain comprises about 12 to about 14 amino acids; preferably wherein X₃ comprises at least two consecutive, negatively charged amino acids wherein each negatively charged amino acid is independently selected from Asp and Glu; preferably, the C-terminal domain comprises or consists of the amino acid sequence of SEQ ID NO: 60; or alternatively, wherein the C-terminal domain of Formula 2 comprises or consists of the amino acid sequence of SEQ ID NO: 49.

In certain aspects, the N-1 domain has at least about 85%, at least about 90%, at least about 95%, at least about 97%, at least about 98%, or at least about 99% sequence identity to one of SEQ ID NOs: 7, 8, 45, 46, and 69.

### Therapeutic Uses

Inhibition of β-Catenin Nuclear Translocation and/or Signaling Pathway

The discovery that the Dkk3 gene locus encodes a second gene product, DKK3b, a vital intracellular protein that directly regulates β-catenin trafficking resolves the longstanding confusion about the molecular function of this important component of the β-catenin signaling pathway. DKK3b provides a new level of regulation in the β-catenin signaling pathway that is independent of the Wnt ligand and is essential for embryogenesis. DKK3b is located downstream of the Wnt regulated degradation complex where it regulates β-catenin trafficking to the nucleus and has the capacity to protect β-catenin from proteolysis by redirecting it to the actin cytoskeleton. DKK3b rapidly shuttles between the perinuclear space and the cytoplasmic surface of the plasma membrane in astrocytes using myosin motors and actin fibers. This intracellular cycling by DKK3b may provide a functional shuttling service capable of relocating β-catenin from the vicinity of the nucleus back to its plasma membrane reservoir, closing a previously unrecognized arm of the regulatory loop. DKK3b is an essential component of the Wnt/β-catenin pathway and directly antagonizes the pro-proliferative β-catenin signaling molecule providing an important new point of control that impacts the regulatory pathways responsible for differentiation, lineage specification, pluripotency and oncogenesis.

DKK3b also acts more broadly to regulate other β-TrCP target substrates in addition to β-catenin, including NF-kB, p38, Decaptor, and Erk1/2. This adds a new dimension of regulation to one of the most studied ubiquitin-proteasome systems (UPS) in the cell.

As a modulator of β-TrCP substrate degradation and nuclear entry, DKK3b is an attractive target for the creation of new drugs for intervention in β-catenin stabilization and subsequent translocation to the nucleus which is often a key step that is dysregulated in various β-catenin-related diseases as varied as cancer/proliferative, metabolic, osteoporosis, neurological, immunological, endocrinologic, cardiovascular, hematologic, and diabetes.

Therefore, the invention provides methods of inhibiting β-catenin nuclear translocation or a β-catenin signaling pathway comprising administering a therapeutically effective amount of a peptide mimetic of the invention to a patient suffering from a β-catenin-related disease.

### Cancer Treatment

Preferably the invention provides compositions and methods for the treatment of cancer by inhibition of β-catenin nuclear translocation or of a β-catenin signaling pathway. Compositions comprising the peptide mimetics of the invention are useful in the treatment of many types of cancer. The invention provides methods of administering a therapeutically effective amount of a pharmaceutical compositions comprising a peptide mimetic of the invention to a cancer patient in need thereof. Pharmaceutical compositions, dosing, and combination therapies for treating cancer are described herein.

The term “cancer”, as used herein, shall be given its ordinary meaning, as a general term for diseases in which abnormal cells divide without control. In particular, and in the context of the embodiments of the present invention, cancer refers to angiogenesis-related cancer. Cancer cells can invade nearby tissues and can spread through the bloodstream and lymphatic system to other parts of the body. There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Glioblastoma, Childhood; Glioblastoma, Adult; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland' Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth), and continue their rapid growth and division in this new location. This is called metastasis. Once malignant cells have metastasized, achieving a cure is more difficult. Benign tumors have less of a tendency to invade and are less likely to metastasize.

Preferably, cancers that may be treated with the compositions and methods described herein include, but are not limited to: melanoma (e.g., metastatic malignant melanoma), renal cancer (e.g. clear cell carcinoma), prostate cancer (e.g., hormone refractory prostate adenocarcinoma), pancreatic cancer (e.g., adenocarcinoma), breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies.

Preferably the compositions and methods of the invention are used for treating solid tumors including but not limited to lymphomas, melanoma, renal cell carcinoma (RCC), advanced solid tumors, tumors that have previously been treated with therapeutic therapy but remain refractory to previous therapies. Therefore, the invention includes reducing a tumor in a cancer patient comprising administering to the patient a therapeutically effective amount of a peptide mimetic of the invention.

Preferably the compositions of the invention are used to reduce tumors in a cancer patient. The term “reducing a tumor” as used herein refers to a reduction in the size or volume of a tumor mass, a decrease in the number of metastasized tumors in a subject, a decrease in the proliferative status (the degree to which the cancer cells are multiplying) of the cancer cells, and the like.

### Combination Therapy for Cancer Treatment

While the peptide mimetics of the invention may be used in cancer treatment as a monotherapy, the combination of a peptide mimetic with other therapeutic anticancer treatments in the context of the invention is also contemplated. Therefore, the methods of the invention comprise administering at least one peptide mimetic of the in invention in combination with one or more anticancer agents and their associated anticancer therapeutic treatment regimens. The terms “agent”, “anticancer therapeutic”, “anticancer agent” and “therapeutic” maybe used interchangeably herein and collectively refer to compounds and molecules that have anti-cancer properties or are otherwise useful in the treatment of cancer and in cancer treatment regimens.

Other therapeutic anticancer agents and associated therapeutic anticancer treatment regimens include immunotherapies such as adoptive cell transfer regimens, antigen-specific vaccination, inhibition of DNA repair proteins (e.g., inhibitors of the nucleic enzyme poly(adenosine 5′-diphospho-ribose) polymerase “poly(ADP-ribose) polymerase” (“PARP inhibitors”) and blockade of immune checkpoint inhibitory molecules, for example cytotoxic T lymphocyte-associated antigen 4 (CTLA-4) programmed death 1 (PD-1) antibodies such as pembrolizumab and nivolumab.

Other co-therapeutic anti-cancer treatment regimens include combinations with chemotherapeutic agents including but not limited to, alkylating agents, antitumor antibiotics, antimetabolic agents, other anti-tumor antibiotics, and plant derived agents, small molecules that are effective in treating cancer are well known in the art and include antagonists of factors that are involved in tumor growth, such as EGFR, ErbB2 (also known as Her2) ErbB3, ErbB4, or TNF, therapeutic proteins for treating cancer such as a suicide protein that causes cell death by itself or in the presence of other compounds, and therapeutic antibodies such as trastuzumab, bevacizumab, rituximab.

“Immune checkpoint proteins” regulate T cell function in the immune system. T cells play a central role in cell-mediated immunity. Checkpoint proteins interact with specific ligands that send a signal into the T cell and essentially switch off or inhibit T cell function. Cancer cells take advantage of this system by driving high levels of expression of checkpoint proteins on their surface that results in control of the T cells expressing checkpoint proteins on the surface of T cells that enter the tumor microenvironment, thus suppressing the anticancer immune response. As such, inhibition of checkpoint proteins by agents referred to herein as “immune checkpoint protein (ICP) inhibitors” would result in restoration of T cell function and an immune response to the cancer cells. Examples of checkpoint proteins include, but are not limited to: CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, OX40, B-7 family ligands or a combination thereof. Preferably, the immune checkpoint inhibitor interacts with a ligand of a checkpoint protein which may be CTLA-4, PDL1, PDL2, PD1, B7-H3, B7-H4, BTLA, HVEM, GAL9, LAG3, VISTA, KIR, 2B4, CD160, CGEN-15049, CHK 1, CHK2, OX40, A2aR, B-7 family ligands or a combination thereof. Preferably, the checkpoint inhibitor is a biologic therapeutic or a small molecule. Preferably, the checkpoint inhibitor is a monoclonal antibody, a humanized antibody, a fully human antibody, a peptide mimetic or a combination thereof. Preferably, the PD1 checkpoint inhibitor comprises one or more anti-PD-1 antibodies, including nivolumab and pembrolizumab.

Treatment regimens with the peptide mimetic in accordance with the invention may also be combined with other therapeutic agents for treating cancer. Preferably, the therapeutic agent and/or anti-cancer agent is an antibody. Preferably, the therapeutic agent is a therapeutic protein. Preferably, the therapeutic agent is a small molecule. Preferably the anticancer agent is an antigen. Preferably, the therapeutic agent is a population of cells. Preferably, the therapeutic agent is a therapeutic antibody. Preferably the therapeutic agent is another cytotoxic and/or chemotherapeutic agent. The term “cytotoxic agent” as used herein refers to a substance that inhibits or prevents a cellular function and/or causes cell death or destruction. Chemotherapeutic agent” includes chemical compounds useful in the treatment of cancer.

### Antibodies

Preferably the peptide mimetics of the invention are combined with a therapeutic antibody. Methods of producing antibodies, and antigen-binding fragments thereof, are well known in the art and are disclosed in, e.g., U.S. Pat. No. 7,247,301, US2008/0138336, and U.S. Pat. No. 7,923,221, all of which are herein incorporated by reference in their entirety. Therapeutic antibodies that can be used in the methods of the present invention include, but are not limited to, any of the art-recognized therapeutic antibodies that are approved for use, in clinical trials, or in development for clinical use. In some embodiments, more than one therapeutic antibody can be included in the combination therapy of the present invention. Non-limiting examples of therapeutic antibodies include the following, without limitation:
trastuzumab (HERCEPTIN™ by Genentech, South San Francisco, Calif.), which is used to treat HER-2/neu positive breast cancer or metastatic breast cancer; bevacizumab (AVASTIN™ by Genentech), which is used to treat colorectal cancer, metastatic colorectal cancer, breast cancer, metastatic breast cancer, non-small cell lung cancer, or renal cell carcinoma;
rituximab (RITUXAN™ by Genentech), which is used to treat non-Hodgkin's lymphoma or chronic lymphocytic leukemia;
pertuzumab (OMNITARG™ by Genentech), which is used to treat breast cancer, prostate cancer, non-small cell lung cancer, or ovarian cancer;
cetuximab (ERBITUX™ by ImClone Systems Incorporated, New York, N.Y.), which can be used to treat colorectal cancer, metastatic colorectal cancer, lung cancer, head and neck cancer, colon cancer, breast cancer, prostate cancer, gastric cancer, ovarian cancer, brain cancer, pancreatic cancer, esophageal cancer, renal cell cancer, prostate cancer, cervical cancer, or bladder cancer;
IMC-1C11 (ImClone Systems Incorporated), which is used to treat colorectal cancer, head and neck cancer, as well as other potential cancer targets;
tositumomab and tositumomab and iodine I¹³¹(BEXXAR™ by Corixa Corporation, Seattle, Wash.), which is used to treat non-Hodgkin's lymphoma, which can be CD20 positive, follicular, non-Hodgkin's lymphoma, with and without transformation, whose disease is refractory to Rituximab and has relapsed following chemotherapy;
In¹¹¹ ibirtumomab tiuxetan; Y⁹⁰ ibirtumomab tiuxetan; I¹¹¹ ibirtumomab tiuxetan and Y⁹⁰ ibirtumomab tiuxetan (ZEVALIN™ by Biogen Idec, Cambridge, Mass.), which is used to treat lymphoma or non-Hodgkin's lymphoma, which can include relapsed follicular lymphoma; relapsed or refractory, low grade or follicular non-Hodgkin's lymphoma; or transformed B-cell non-Hodgkin's lymphoma;
EMD 7200 (EMD Pharmaceuticals, Durham, N.C.), which is used for treating for treating non-small cell lung cancer or cervical cancer;
SGN-30 (a genetically engineered monoclonal antibody targeted to CD30 antigen by Seattle Genetics, Bothell, Wash.), which is used for treating Hodgkin's lymphoma or non-Hodgkin's lymphoma;
SGN-15 (a genetically engineered monoclonal antibody targeted to a Lewisy-related antigen that is conjugated to doxorubicin by Seattle Genetics), which is used for treating non-small cell lung cancer;
SGN-33 (a humanized antibody targeted to CD33 antigen by Seattle Genetics), which is used for treating acute myeloid leukemia (AML) and myelodysplastic syndromes (MDS);
SGN-40 (a humanized monoclonal antibody targeted to CD40 antigen by Seattle Genetics), which is used for treating multiple myeloma or non-Hodgkin's lymphoma;
SGN-35 (a genetically engineered monoclonal antibody targeted to a CD30 antigen that is conjugated to auristatin E by Seattle Genetics), which is used for treating non-Hodgkin's lymphoma;
SGN-70 (a humanized antibody targeted to CD70 antigen by Seattle Genetics), that is used for treating renal cancer and nasopharyngeal carcinoma;
SGN-75 (a conjugate comprised of the SGN70 antibody and an Auristatin derivative by Seattle Genetics); and
SGN-17/19 (a peptide mimetic containing antibody and enzyme conjugated to melphalan prodrug by Seattle Genetics), which is used for treating melanoma or metastatic melanoma.

The therapeutic antibodies to be used in the methods of the present invention are not limited to those described herein. For example, the following approved therapeutic antibodies can also be used in the methods of the invention: brentuximab vedotin (ADCETRIS™) for anaplastic large cell lymphoma and Hodgkin lymphoma, ipilimumab (MDX-101; YERVOY™) for melanoma, ofatumumab (ARZERRA™) for chronic lymphocytic leukemia, panitumumab (VECTIBIX™) for colorectal cancer, alemtuzumab (CAMPATH™) for chronic lymphocytic leukemia, ofatumumab (ARZERRA™) for chronic lymphocytic leukemia, gemtuzumab ozogamicin (MYLOTARG™) for acute myelogenous leukemia.

Antibodies for use in accordance with the invention can also target molecules expressed by immune cells, such as, but not limited to, tremelimumab (CP-675,206) and ipilimumab (MDX-010) which targets CTLA4 and has the effect of tumor rejection, protection from re-challenge, and enhanced tumor-specific T cell responses; OX86 which targets OX40 and increases antigen-specific CD8+ T cells at tumor sites and enhances tumor rejection; CT-011 which targets PD 1 and has the effect of maintaining and expanding tumor specific memory T cells and activates NK cells; BMS-663513 which targets CD137 and causes regression of established tumors, as well as the expansion and maintenance of CD8+ T cells, and daclizumab (ZENAPAX™) which targets CD25 and causes transient depletion of CD4+CD25+FOXP3+Tregs and enhances tumor regression and increases the number of effector T cells. A more detailed discussion of these antibodies can be found in, e.g., Weiner et al., Nature Rev. Immunol 2010; 10:317-27.

The therapeutic antibody can be a fragment of an antibody; a complex comprising an antibody; or a conjugate comprising an antibody. The antibody can optionally be chimeric or humanized or fully human.

### Peptides and Proteins

Preferably the methods of the invention include administration of a peptide mimetic of the invention in combination with a therapeutic protein or peptide. Therapeutic proteins that are effective in treating cancer are well known in the art, Preferably, the therapeutic polypeptide or protein is a “suicide protein” that causes cell death by itself or in the presence of other compounds.

A representative example of such a suicide protein is thymidine kinase of the herpes simplex virus. Additional examples include thymidine kinase of varicella zoster virus, the bacterial gene cytosine deaminase (which converts 5-fluorocytosine to the highly toxic compound 5-fluorouracil), p450 oxidoreductase, carboxypeptidase G2, beta-glucuronidase, penicillin-V-amidase, penicillin-G-amidase, beta-lactamase, nitroreductase, carboxypeptidase A, linamarase (also referred to as β-glucosidase), the E. coli gpt gene, and the E. coli Deo gene, although others are known in the art. In some embodiments, the suicide protein converts a prodrug into a toxic compound.

As used herein, “prodrug” means any compound useful in the methods of the present invention that can be converted to a toxic product, i.e. toxic to tumor cells. The prodrug is converted to a toxic product by the suicide protein. Representative examples of such prodrugs include: ganciclovir, acyclovir, and FIAU (1-(2-deoxy-2-fluoro-β-D-arabinofuranosyl)-5-iod-ouracil) for thymidine kinase; ifosfamide for oxidoreductase; 6-methoxypurine arabinoside for VZV-TK; 5-fluorocytosine for cytosine deaminase; doxorubicin for beta-glucuronidase; CB 1954 and nitrofurazone for nitroreductase; and N-(Cyanoacetyl)-L-phenylalanine or N-(3-chloropropionyl)-L-phenylalanine for carboxypeptidase A. The prodrug may be administered readily by a person having ordinary skill in this art. A person with ordinary skill would readily be able to determine the most appropriate dose and route for the administration of the prodrug.

Preferably the therapeutic protein or polypeptide, is a cancer suppressor, for example p53 or Rb, or a nude acid encoding such a protein or polypeptide. Those of skill know of a wide variety of such cancer suppressors and how to obtain them and/or the nucleic acids encoding them.

Other examples of anti-cancer/therapeutic proteins or polypeptides include pro-apoptotic therapeutic proteins and polypeptides, for example, p15, p16, or p21^{WAF-1}.

Cytokines, and nucleic acid encoding them may also be used as therapeutic proteins and polypeptides. Examples include: GM-CSF (granulocyte macrophage colony stimulating factor); TNF-alpha (Tumor necrosis factor alpha); Interferons including, but not limited to, IFN-alpha and IFN-gamma; and Interleukins including, but not limited to, Interleukin-1 (IL-1), Interleukin-Beta (IL-beta), Interleukin-2 (IL-2), Interleukin-4 (IL-4), Interleukin-5 (IL-5), Interleukin-6 (IL-6), Interleukin-7 (IL-7), Interleukin-8 (IL-8), Interleukin-10 (IL-10), Interleukin-12 (IL-12), Interleukin-13 (IL-13), Interleukin-14 (IL-14), Interleukin-15 (IL-15), Interleukin-16 (IL-16), Interleukin-18 (IL-18), Interleukin-23 (IL-23), Interleukin-24 (IL-24), although other embodiments are known in the art.

Additional examples of cytocidal genes includes, but is not limited to, mutated cyclin G1 genes. By way of example, the cytocidal gene may be a dominant negative mutation of the cyclin G1 protein (e.g., WO/01/64870).

### Vaccines

Preferably the methods of the invention include administration of a peptide mimetic of the invention in combination with a cancer vaccine for stimulating a cancer specific-immune response, e.g., innate and adaptive immune responses, for generating host immunity against a cancer. Illustrative vaccines include, but are not limited to, for example, antigen vaccines, whole cell vaccines, dendritic cell vaccines, and DNA vaccines. Depending upon the particular type of vaccine, the vaccine composition may include one or more suitable adjuvants known to enhance a subject's immune response to the vaccine.

The vaccine may, for example, be cellular based, i.e., created using cells from the patient's own cancer cells to identify and obtain an antigen. Exemplary vaccines include tumor cell-based and dendritic-cell based vaccines, where activated immune cells from the subject are delivered back to the same subject, along with other proteins, to further facilitate immune activation of these tumor antigen primed immune cells. Tumor cell-based vaccines include whole tumor cells and gene-modified tumor cells. Whole tumor cell vaccines may optionally be processed to enhance antigen presentation, e.g., by irradiation of either the tumor cells or tumor lysates). Vaccine administration may also be accompanied by adjuvants such as bacillus calmette-guerin (BCG) or keyhole limpet hemocyanin (KLH), depending upon the type of vaccine employed. Plasmid DNA vaccines may also be used and can be administered via direct injection or biolistically. Also contemplated for use are peptide vaccines, viral gene transfer vector vaccines, and antigen-modified dendritic cells (DCs).

Preferably the vaccine is a therapeutic cancer peptide-based vaccine. Peptide vaccines can be created using known sequences or from isolated antigens from a subject's own tumor(s) and include neoantigens and modified antigens. Illustrative antigen-based vaccines include those where the antigen is a tumor-specific antigen. For example, the tumor-specific antigen may be selected from a cancer-testis antigen, a differentiation antigen, and a widely occurring over-expressed tumor associated antigen, among others. Recombinant peptide vaccines, based on peptides from tumor-associated antigens, when used in the instant method, may be administered or formulated with, an adjuvant or immune modulator. Illustrative antigens for use in a peptide-based vaccine include, but are not limited to, the following, since this list is meant to be purely illustrative. For example, a peptide vaccine may comprise a cancer-testis antigen such as MAGE, BAGE, NY-ESO-1 and SSX-2, encoded by genes that are normally silenced in adult tissues but transcriptionally reactivated in tumor cells. Alternatively, the peptide vaccine may comprise a tissue differentiation associated antigen, i.e., an antigen of normal tissue origin and shared by both normal and tumorous tissue. For example, the vaccine may comprise a melanoma-associated antigen such as gp100, Melan-A/Mart-1, MAGE-3, or tyrosinase; or may comprise a prostate cancer antigen such as PSA or PAP. The vaccine may comprise a breast cancer-associated antigen such as mammaglobin-A. Other tumor antigens that may be comprised in a vaccine for use in the instant method include, for example, CEA, MUC-1, HER1/Nue, hTERT, ras, and B-raf. Other suitable antigens that may be used in a vaccine include SOX-2 and OCT-4, associated with cancer stem cells or the EMT process.

Antigen vaccines include multi-antigen and single antigen vaccines. Exemplary cancer antigens may include peptides having from about 5 to about 30 amino acids, or from about 6 to 25 amino acids, or from about 8 to 20 amino acids.

As described above, an immunostimulatory adjuvant (different from RSLAIL-2) may be used in a vaccine, in particular, a tumor-associated antigen-based vaccine, to assist in generating an effective immune response. For example, a vaccine may incorporate a pathogen-associated molecular pattern (PAMP) to assist in improving immunity. Additional suitable adjuvants include monophosphoryl lipid A, or other lipopolysaccharides; toll-like receptor (TLR) agonists such as, for example, imiquimod, resiquimod (R-848), TLR3, IMO-8400, and rintatolimod. Additional adjuvants suitable for use include heat shock proteins.

A genetic vaccine typically uses viral or plasmid DNA vectors carrying expression cassettes. Upon administration, they transfect somatic cells or dendritic cells as part of the inflammatory response to thereby result in cross-priming or direct antigen presentation. Preferably, a genetic vaccine is one that provides delivery of multiple antigens in one immunization. Genetic vaccines include DNA vaccines, RNA vaccines and viral-based vaccines.

DNA vaccines for use in the instant methods are bacterial plasmids that are constructed to deliver and express tumor antigen. DNA vaccines may be administered by any suitable mode of administration, e.g., subcutaneous or intradermal injection, but may also be injected directly into the lymph nodes. Additional modes of delivery include, for example, gene gun, electroporation, ultrasound, laser, liposomes, microparticles and nanoparticles.

Preferably, the vaccine comprises a neoantigen, or multiple neoantigens. Preferably, the vaccine is a neoantigen-based vaccine. Preferably a neoantigen-based vaccine (NBV) composition may encode multiple cancer neoantigens in tandem, where each neoantigen is a polypeptide fragment derived from a protein mutated in cancer cells. For instance, a neoantigenic vaccine may comprise a first vector comprising a nucleic acid construct encoding multiple immunogenic polypeptide fragments, each of a protein mutated in cancer cells, where each immunogenic polypeptide fragment comprises one or more mutated amino acids flanked by a variable number of wild type amino acids from the original protein, and each polypeptide fragment is joined head-to-tail to form an immunogenic polypeptide. The lengths of each of the immunogenic polypeptide fragments forming the immunogenic polypeptide can vary.

Viral gene transfer vector vaccines may also be used; in such vaccines, recombinant engineered virus, yeast, bacteria or the like is used to introduce cancer-specific proteins to the patient's immune cells. In a vector-based approach, which can be tumor lytic or non-tumor lytic, the vector can increase the efficiency of the vaccine due to, for example, its inherent immunostimulatory properties. Illustrative viral-based vectors include those from the poxviridae family, such as vaccinia, modified vaccinia strain Ankara and avipoxviruses. Also suitable for use is the cancer vaccine, PROSTVAC, containing a replication-competent vaccinia priming vector and a replication-incompetent fowlbox-boosting vector. Each vector contains transgenes for PSA and three co-stimulatory molecules, CD80, CD54 and CD58, collectively referred to as TRICOM. Other suitable vector-based cancer vaccines include Trovax and TG4010 (encoding MUC1 antigen and IL-2). Additional vaccines for use include bacteria and yeast-based vaccines such as recombinant Listeria monocytogenes and Saccharomyces cerevisae.

The foregoing vaccines may be combined and/or formulated with adjuvants and other immune boosters to increase efficacy. Depending upon the particular vaccine, administration may be either intratumoral or non-intratumoral (i.e., systemic).

### Small Molecules

Preferably, the methods of the invention include coadministration of a peptide mimetic of the invention in combination with an anticancer small molecule. Small molecules that are effective in treating cancer are well known in the art and include antagonists of factors that are involved in tumor growth, such as EGFR, ErbB2 (also known as Her2) ErbB3, ErbB4, or TNF. Non-limiting examples include small molecule receptor tyrosine kinase inhibitors (RTKIs) that target one or more tyrosine kinase receptors, such as VEGF receptors, FGF receptors, EGF receptors and PDGF receptors.

Many therapeutic small molecule RTKIs are known in the art, including, but are not limited to, vatalanib (PTK787), erlotinib (TARCEVA™), OSI-7904, ZD6474 (ZACTIMA™), ZD6126 (ANG453), ZD1839, sunitinib (SUTENT™), semaxanib (SU5416), AMG706, AG013736, Imatinib (GLEEVEC™), MLN-518, CEP-701, PKC-412, Lapatinib (GSK572016), VELCADE™, AZD2171, sorafenib (NEXAVAR™), XL880, and CHIR-265. Small molecule protein tyrosine phosphatase inhibitors, such as those disclosed in Jiang et al., Cancer Metastasis Rev. 2008; 27:263-72 are also useful for practicing the methods of the invention. Such inhibitors can target, e.g., HSP2, PRL, PTP1B, or Cdc25 phosphatases.

Small molecules that target Bc1-2/Bc1-XL, such as those disclosed in US2008/0058322, are also useful for practicing the methods of the present invention. Further exemplary small molecules for use in the present invention are disclosed in Zhang et al. Nature Reviews: Cancer 2009; 9:28-39. In particular, chemotherapeutic agents that lead to immunogenic cell death such as anthracyclins (Kepp et al., Cancer and Metastasis Reviews 2011; 30:61-9) will be well suited for synergistic effects with extended-PK IL-2.

### Additional Cancer Antigens and Vaccines

Preferably, the methods of the invention include administration of a peptide mimetic of the invention in combination with a cancer antigen, e.g., for use as a cancer vaccine (see, e.g., Overwijk, et al. Journal of Experimental Medicine 2008; 198:569-80). Other cancer antigens that can be used in vaccinations include, but are not limited to, (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

The cancer antigen may be an epithelial cancer antigen, (e.g., breast, gastrointestinal, lung), a prostate specific cancer antigen (PSA) or prostate specific membrane antigen (PSMA), a bladder cancer antigen, a lung (e.g., small cell lung) cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, or a colorectal cancer antigen.

In another embodiment, the cancer antigen is a lymphoma antigen (e.g., non-Hodgkin's lymphoma or Hodgkin's lymphoma), a B-cell lymphoma cancer antigen, a leukemia antigen, a myeloma (i.e., multiple myeloma or plasma cell myeloma) antigen, an acute lymphoblastic leukemia antigen, a chronic myeloid leukemia antigen, or an acute myelogenous leukemia antigen. The described cancer antigens are only exemplary, and that any cancer antigen can be targeted in the present invention.

Preferably, the cancer antigen is a mucin-1 protein or peptide (MUC-1) that is found on all human adenocarcinomas: pancreas, colon, breast, ovarian, lung, prostate, head and neck, including multiple myelomas and some B cell lymphomas. Patients with inflammatory bowel disease, either Crohn's disease or ulcerative colitis, are at an increased risk for developing colorectal carcinoma. MUC-1 is a type I transmembrane glycoprotein. The major extracellular portion of MUC-1 has a large number of tandem repeats consisting of 20 amino acids which comprise immunogenic epitopes. In some cancers it is exposed in an unglycosylated form that is recognized by the immune system (Gendler et al., J Biol Chem 1990; 265:15286-15293).

In another embodiment, the cancer antigen is a mutated B-Raf antigen, which is associated with melanoma and colon cancer. The vast majority of these mutations represent a single nucleotide change of T-A at nucleotide 1796 resulting in a valine to glutamic acid change at residue 599 within the activation segment of B-Raf. Raf proteins are also indirectly associated with cancer as effectors of activated Ras proteins, oncogenic forms of which are present in approximately one-third of all human cancers. Normal non-mutated B-Raf is involved in cell signaling, relaying signals from the cell membrane to the nucleus. The protein is usually only active when needed to relay signals. In contrast, mutant B-Raf has been reported to be constantly active, disrupting the signaling relay (Mercer and Pritchard, Biochim Biophys Acta (2003) 1653(1):25-40; Sharkey et al., Cancer Res. (2004) 64(5):1595-1599).

Preferably, the cancer antigen is a human epidermal growth factor receptor-2 (HER-2/neu) antigen. Cancers that have cells that overexpress HER-2/neu are referred to as HER-2/neu⁺ cancers. Exemplary HER-2/neu⁺ cancers include prostate cancer, lung cancer, breast cancer, ovarian cancer, pancreatic cancer, skin cancer, liver cancer (e.g., hepatocellular adenocarcinoma), intestinal cancer, and bladder cancer.

HER-2/neu has an extracellular binding domain (ECD) of approximately 645 aa, with 40% homology to epidermal growth factor receptor (EGFR), a highly hydrophobic transmembrane anchor domain (TMD), and a carboxyterminal intracellular domain (ICD) of approximately 580 aa with 80% homology to EGFR. The nucleotide sequence of HER-2/neu is available at GENBANK™. Accession Nos. AH002823 (human HER-2 gene, promoter region and exon 1); M16792 (human HER-2 gene, exon 4): M16791 (human HER-2 gene, exon 3); M16790 (human HER-2 gene, exon 2); and M16789 (human HER-2 gene, promoter region and exon 1). The amino acid sequence for the HER-2/neu protein is available at GENBANK™. Accession No. AAA58637. Based on these sequences, one skilled in the art could develop HER-2/neu antigens using known assays to find appropriate epitopes that generate an effective immune response.

Exemplary HER-2/neu antigens include p369-377 (a HER-2/neu derived HLA-A2 peptide); dHER2 (Corixa Corporation); li-Key MHC class II epitope hybrid (Generex Biotechnology Corporation); peptide P4 (amino acids 378-398); peptide P7 (amino acids 610-623); mixture of peptides P6 (amino acids 544-560) and P7; mixture of peptides P4, P6 and P7; HER2 [9₇₅₄]; and the like.

Preferably, the cancer antigen is an epidermal growth factor receptor (EGFR) antigen. The EGFR antigen can be an EGFR variant 1 antigen, an EGFR variant 2 antigen, an EGFR variant 3 antigen and/or an EGFR variant 4 antigen. Cancers with cells that overexpress EGFR are referred to as EGFR cancers. Exemplary EGFR cancers include lung cancer, head and neck cancer, colon cancer, colorectal cancer, breast cancer, prostate cancer, gastric cancer, ovarian cancer, brain cancer and bladder cancer.

Preferably, the cancer antigen is a vascular endothelial growth factor receptor (VEGFR) antigen. VEGFR is considered to be a regulator of cancer-induced angiogenesis. Cancers with cells that overexpress VEGFR are called VEGFR⁺ cancers. Exemplary VEGFR⁺ cancers include breast cancer, lung cancer, small cell lung cancer, colon cancer, colorectal cancer, renal cancer, leukemia, and lymphocytic leukemia.

Preferably, the cancer antigen is prostate-specific antigen (PSA) and/or prostate-specific membrane antigen (PSMA) that are prevalently expressed in androgen-independent prostate cancers.

Preferably, the cancer antigen is Gp-100 Glycoprotein 100 (gp 100) is a tumor-specific antigen associated with melanoma.

Preferably, the cancer antigen is a carcinoembryonic (CEA) antigen. Cancers with cells that overexpress CEA are referred to as CEA⁺ cancers. Exemplary CEA⁺ cancers include colorectal cancer, gastric cancer and pancreatic cancer. Exemplary CEA antigens include CAP-1 (i.e., CEA aa 571-579), CAP1-6D, CAP-2 (i.e., CEA aa 555-579), CAP-3 (i.e., CEA aa 87-89), CAP-4 (CEA aa 1-11), CAP-5 (i.e., CEA aa 345-354), CAP-6 (i.e., CEA aa 19-28) and CAP-7.

Preferably, the cancer antigen is carbohydrate antigen 10.9 (CA 19.9). CA 19.9 is an oligosaccharide related to the Lewis A blood group substance and is associated with colorectal cancers.

Preferably, the cancer antigen is a melanoma cancer antigen. Melanoma cancer antigens are useful for treating melanoma. Exemplary melanoma cancer antigens include MART-1 (e.g., MART-1 26-35 peptide, MART-1 27-35 peptide); MART-1/Melan A; pMel17; pMel17/gp100; gp100 (e.g., gp 100 peptide 280-288, gp 100 peptide 154-162, gp 100 peptide 457-467); TRP-1; TRP-2; NY-ESO-1; p16; beta-catenin; mum-1; and the like.

Preferably, the cancer antigen is a mutant or wild type ras peptide. The mutant ras peptide can be a mutant K-ras peptide, a mutant N-ras peptide and/or a mutant H-ras peptide. Mutations in the ras protein typically occur at positions 12 (e.g., arginine or valine substituted for glycine), 13 (e.g., asparagine for glycine), 61 (e.g., glutamine to leucine) and/or 59. Mutant ras peptides can be useful as lung cancer antigens, gastrointestinal cancer antigens, hepatoma antigens, myeloid cancer antigens (e.g., acute leukemia, myelodysplasia), skin cancer antigens (e.g., melanoma, basal cell, squamous cell), bladder cancer antigens, colon cancer antigens, colorectal cancer antigens, and renal cell cancer antigens.

In another embodiment of the invention, the cancer antigen is a mutant and/or wildtype p53 peptide. The p53 peptide can be used as colon cancer antigens, lung cancer antigens, breast cancer antigens, hepatocellular carcinoma cancer antigens, lymphoma cancer antigens, prostate cancer antigens, thyroid cancer antigens, bladder cancer antigens, pancreatic cancer antigens and ovarian cancer antigens.

The cancer antigen can be a cell, a protein, a peptide, a fusion protein, DNA encoding a peptide or protein, RNA encoding a peptide or protein, a glycoprotein, a lipoprotein, a phosphoprotein, a carbohydrate, a lipopolysaccharide, a lipid, a chemically linked combination of two or more thereof, a fusion or two or more thereof, or a mixture of two or more thereof. In another embodiment, the cancer antigen is a peptide comprising about 6 to about 24 amino acids; from about 8 to about 20 amino acids; from about 8 to about 12 amino acids; from about 8 to about 10 amino acids; or from about 12 to about 20 amino acids. In one embodiment, the cancer antigen is a peptide having an MHC Class I binding motif or a MHC Class II binding motif In another embodiment, the cancer antigen comprises a peptide that corresponds to one or more cytotoxic T lymphocyte (CTL) epitopes.

### Cell Therapy

Preferably, the methods of the invention include administration of a peptide mimetic of the invention in combination with a therapeutic cell therapy. Cell therapies that are useful for treating cancer are well known and are disclosed in, e.g., U.S. Pat. No. 7,402,431. In a preferred embodiment, the cell therapy is T cell transplant. In a preferred method, T cells are expanded ex vivo with IL-2 prior to transplantation into a subject. Methods for cell therapies are disclosed in, e.g., U.S. Pat. No. 7,402,431, US2006/0057121, U.S. Pat. Nos. 5,126,132, 6,255,073, 5,846,827, 6,251,385, 6,194,207, 5,443,983, 6,040,177, 5,766,920, and US2008/0279836.

### Chemotherapy

Preferably the methods of the invention include administration of the peptide mimetic of the invention in combination with chemotherapeutic agents including but not limited to, alkylating agents, antitumor antibiotics, antimetabolic agents, other anti-tumor antibiotics, and plant derived agents.

Alkylating agents are drugs which impair cell function by forming covalent bonds with amino, carboxyl, sulfhydryl and phosphate groups in biologically important molecules. The most important sites of alkylation are DNA, RNA and proteins. Alkylating agents depend on cell proliferation for activity but are not cell-cycle-phase-specific. Alkylating agents suitable for use in the present invention include, but are not limited to, bischloroethylamines (nitrogen mustards, e.g. chlorambucil, cyclophosphamide, ifosfamide, mechlorethamine, melphalan, uracil mustard), aziridines (e.g., thiotepa), alkyl alkone sulfonates (e.g. busulfan), nitroso-ureas (e. g. BCNU, carmustine, lomustine, streptozocin), nonclassic alkylating agents (e.g., altretamine, dacarbazine, and procarbazine), and platinum compounds (e.g., carboplastin, oxaliplatin and cisplatin).

Antitumor antibiotics like adriamycin intercalate DNA at guanine-cytosine and guanine-thymine sequences, resulting in spontaneous oxidation and formation of free oxygen radicals that cause strand breakage. Other antibiotic agents suitable for use in the present invention include, but are not limited to, anthracyclines (e. g. doxorubicin, daunorubicin, epirubicin, idarubicin and anthracenedione), mitomycin C, bleomycin, dactinomycin, and plicatomycin.

Antimetabolic agents suitable for use in the present invention include but are not limited to, floxuridine, fluorouracil, methotrexate, leucovorin, hydroxyurea, thioguanine, mercaptopurine, cytarabine, pentostatin, fludarabine phosphate, cladribine, asparaginase, and gemcitabine.

Plant derived agents include taxanes, which are semisynthetic derivatives of extracted precursors from the needles of yew plants. These drugs have a novel 14-member ring, the taxane. Unlike the vinca alkaloids, which cause microtubular disassembly, the taxanes (e.g., taxol) promote microtubular assembly and stability, therefore blocking the cell cycle in mitosis. Other plant derived agents include, but are not limited to, vincristine, vinblastine, vindesine, vinzolidine, vinorelbine, etoposide, teniposide, and docetaxel.

### Compositions for Combination Therapy

Preferably, the peptide mimetic of the invention is administered together (simultaneously or sequentially) with one or more additional therapeutic agents or other therapeutic agents, such as a therapeutic antibody. Preferably, the peptide mimetic is administered prior to the administration of one or more anticancer therapeutic agents, such as a therapeutic antibody. Preferably, the peptide mimetic is administered concurrent with the administration of one or more anticancer therapeutic agents, such as a therapeutic antibody. Preferably, the peptide mimetic is administered subsequent to the administration of one or more anticancer therapeutic agents, such as a therapeutic antibody. Preferably, the peptide mimetic and one or more therapeutic agents, such as a therapeutic antibody, are administered simultaneously. In other embodiments, the peptide mimetic and one or more therapeutic agents, such as a therapeutic antibody, are administered sequentially. Preferably, the peptide mimetic and one or more therapeutic agents, such as a therapeutic antibody, are administered within one, two, or three days of each other.

The one or more therapeutic agents may be those that serve as adjunctive therapy for cancer, such as cytokines, chemotherapeutic agents, small molecules, antigens, or therapeutic antibodies, and are well known in the art and discussed supra. Additional non-limiting examples of additional agents include GM-CSF (expands monocyte and neutrophil population), IL-7 (important for generation and survival of memory T-cells), interferon alpha, tumor necrosis factor alpha, IL-12, and therapeutic antibodies, such as anti-PD-1, anti-PD-L, anti-CTLA4, anti-CD40, anti-0X40, and anti-CD137, PARP inhibitors, antibodies. In some embodiments, the subject receives the peptide mimetic and one or more therapeutic agents during a same period of prevention, occurrence of a disorder, and/or period of treatment.

Preferably, the invention provides for separate pharmaceutical compositions comprising the peptide mimetic with a pharmaceutically acceptable diluent, carrier, solubilizer, emulsifier, preservative and/or adjuvant and another pharmaceutical composition comprising one or more therapeutic agents, such as a therapeutic antibody, with a pharmaceutically acceptable diluent, carrier, solubilizer, emulsifier, preservative and/or adjuvant.

Preferably, the invention provides for pharmaceutical compositions comprising the peptide mimetic and one or more therapeutic or anti-cancer agents in the same composition, together with a pharmaceutically acceptable diluent, carrier, solubilizer, emulsifier, preservative and/or adjuvant.

### Treatment of Other Diseases

The peptide mimetics of the invention may be useful in treating additional β-catenin-related disorders wherein inhibition of β-catenin nuclear translocation or inhibition of a β-catenin signaling pathway is desired. Such diseases and disorders include, but are not limited to: metabolic diseases, osteoporosis, neurological diseases, immunological diseases, endocrinologic diseases, cardiovascular diseases, hematologic diseases, and inflammatory diseases.

Preferably the invention provides methods of treating cardiovascular disease in a patient comprising administering to the patient a therapeutically effective amount of a peptide mimetic of the invention. “Cardiovascular disease” is defined herein as diseases or disorders affecting heart or blood vessels. Non-limitative examples of cardiovascular diseases or disorders include mostly the acute and chronic manifestation of arteriosclerosis such as acute coronary syndromes, stroke, transient ischemic attacks, arrhythmia, heart failure, and peripheral artery disease. The invention also contemplates combination therapy for treating cardiovascular disease that includes the peptide mimetic of the invention and any other treatment known in the art for treating cardiovascular disease.

Preferably, the invention provides methods of treating an inflammatory disease in a patient comprising administering to the patient a therapeutically effective amount of a peptide mimetic of the invention. Inflammatory diseases or conditions that may be treated with the compositions and methods disclosed herein include any disease or condition characterized by an inflammatory or allergic process as is known in the art, such as inflammation, acute inflammation, chronic inflammation, respiratory disease, atherosclerosis, psoriasis, dermatitis, restenosis, asthma, allergic rhinitis, atopic dermatitis, septic shock, rheumatoid arthritis, inflammatory bowel disease, inflammatory pelvic disease, pain, ocular inflammatory disease, celiac disease, Leigh syndrome, glycerol kinase deficiency, familial eosinophilia, autosomal recessive spastic ataxia, laryngeal inflammatory disease; tuberculosis, chronic cholecystitis, bronchiectasis, silicosis and other pneumoconiosis.

Other diseases and conditions that may be treatable in accordance with the method of the invention include: Ageing, Headaches, Complex Regional Pain Syndrome, Cardiac Hypertrophy, Muscular Dystrophy (type 2A),Catabolic disorders; Diabetes mellitus, Type 1 Diabetes mellitus, Type 2, Fetal Growth Retardation, Hypercholesterolemia, Atherosclerosis, Heart Disease, Chronic Heart Failure, Ischemia/reperfusion, Stroke, Angina Pectoris, Pulmonary Disease, Cystic Fibrosis Pulmonary hypertension, Hyaline Membrane, Kidney Disease, Glomerular Disease, Alcoholic Liver Disease, Leptospirosis, renal disease, Gut Diseases, Peritoneal endometriosis, Skin Diseases, Nasal sinusitis, Anhidrotic Ecodermal Dysplasia-ID, Behcet's Disease, Incontinentia pigmenti, Tuberculosis, Asthma, Arthritis, Crohn's Disease, Colitis, Ocular Allergy, Bielory Glaucoma, Appendicitis, Paget' s Disease, Pancreatitis, Periodonitis, Endometriosis, Inflammatory Bowel Disease, Inflammatory Lung Disease, Sepsis Silica-induced, Sleep apnea, AIDS (HIV-1), Autoimmunity, Antiphospholipid Syndrome, Lupus, Lupus nephritis, Chronic Disease Syndrome, Familial Mediterranean Fever, Hereditary Periodic Fever Syndrome, Psychosocial stress diseases, Neuropathological Diseases, Familial amyloidotic polyneuropathy, inflammatory neuropathy, Traumatic brain injury, Parkinson Disease, Multiple Sclerosis, Rheumatic Disease, Alzheimers Disease, Amyotropic lateral sclerosis (ALS), Huntington's Disease, Retinal Disease, Cataracts, and Hearing loss.

### Pharmaceutical Compositions

The invention provides pharmaceutical compositions comprising one or more of the peptide mimetics of the invention and one or more pharmaceutically acceptable excipients. Pharmaceutical compositions may optionally comprise one or more additional active substances, for example, therapeutically and/or prophylactically active substances. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

Preferably, compositions are administered to humans, human patients or subjects. For the purposes of the present disclosure, the phrase “active ingredient” generally refers to a peptide mimetic of the invention or variants thereof to be delivered as described herein.

Pharmaceutical compositions of the invention can be formulated using one or more excipients. Pharmaceutical compositions described herein may be prepared by any method known or hereafter developed in the art of pharmacology. In general, such preparatory methods include a step associating active ingredients with excipient and/or one or more accessory ingredients.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants, lipidoids, liposomes, lipid nanoparticles, polymers, lipoplexes, core-shell nanoparticles, peptides, proteins, and combinations thereof as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use. General considerations in the formulation and/or manufacture of pharmaceutical agents may be found, for example, in Remington's The Science and Practice of Pharmacy, 21^{st}Edition, A. R. Gennaro (Lippincott, Williams & Wilkins, Baltimore, Md., 2006; incorporated herein by reference).

### Dosing and Administration

The invention provides methods comprising administering peptide mimetics of the invention to a subject in need thereof. Preferably peptide mimetics of the invention and pharmaceutical compositions comprising peptide mimetics of the invention may be administered by any route which results in therapeutically effective outcomes including, but not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops.

The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically or prophylactically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Preferably, compositions in accordance with the invention may be administered at dosage levels sufficient to deliver from about 0.001 mg/kg to about 200 mg/kg, from about 0.001 mg/kg to about 0.01 mg/kg, from about 0.003 mg/kg to about 0.03 mg/kg, from about 0.005 mg/kg to about 0.05 mg/kg, from about 0.015 mg/kg to about 0.15 mg/kg, from about 0.02 mg/kg to about 0.2 mg/kg, from about 0.03 mg/kg to about 0.3 mg/kg, from about 0.05 mg/kg to about 0.5 mg/kg, from about 0.1 mg/kg to about 1 mg/kg, from about 0.15 mg/kg to about 1.5 mg/kg, from about 0.2 mg/kg to about 2 mg/kg, from about 0.3 mg/kg to about 3 mg/kg, from about 5 mg/kg to about 50 mg/kg, from about 10 mg/kg to about 60 mg/kg, from about 15 mg/kg to about 65 mg/kg, from about 20 mg/kg to about 70 mg/kg, or from about 30 mg/kg to about 80 mg/kg, from about 40 mg/kg to about 90 mg/kg, from about 50 mg/kg to about 100 mg/kg, from about 75 mg/kg to about 150 mg/kg, from about 100 mg/kg to about 150 mg/kg or at least 200 mg/kg of subject body weight per day, one or more times a day, to obtain the desired therapeutic, diagnostic or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). The desired dosage may comprise 5 administrations over a 2 week period.

Preferably, pharmaceutical compositions of the invention are administered using a split dose. As used herein, a “split dose” is the division of a single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose.

The pharmaceutical composition may be administered once daily, or may be administered as two, three or more sub-doses at appropriate intervals throughout the day or even using continuous infusion or delivery through a controlled release formulation. Preferably, a peptide mimetic contained in each sub-dose must be correspondingly smaller in order to achieve the total daily dosage. Dosing may also be according to multi-dosing schemes of one, two, three, four, five or more doses.

The dosing may be administered as two, three or more sub-doses at appropriate intervals over a day, more than one day, week, 2 weeks, 3 weeks, 1 month or greater.

The dosage unit may be administered using continuous infusion over an appropriate time interval or delivery may occur through a controlled release formulation. For example, a peptide mimetic can be administered using continuous infusion over 1 minute, 5 minutes, 10 minutes, 15 minutes, 30 minutes, 45 minutes, 1 hour, 2 hours, 3 hours, 4 hours, 5 hours or more. The dosage unit can also be compounded for delivery over several days, e.g., using a conventional sustained release formulation which provides sustained release over a several day period. Sustained release formulations are well known in the art and are particularly useful for delivery of agents at a particular site, such as could be used with the agents of the present invention. In this embodiment, the dosage unit contains a corresponding multiple of the daily dose.

The effect of a single dose on any particular phenotype or symptom can be long lasting, such that subsequent doses are administered at not more than 3, 4, or 5 day intervals, or at not more than 1, 2, 3, or 4 week intervals.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual pharmaceutical compositions encompassed by the invention can be made using conventional methodologies or on the basis of in vivo testing using an appropriate animal model.

### Delivery Systems

Various delivery systems are known and can be used to administer a peptide mimetic and/or pharmaceutical compositions thereof in accordance with the invention, e.g., encapsulation in liposomes, microparticles, microcapsules, recombinant cells capable of expressing the compound, receptor-mediated endocytosis, construction of a nucleic acid as part of a retroviral or other vector, etc. The peptide mimetic or compositions thereof may be administered by infusion or bolus injection, by absorption through epithelial or mucocutaneous linings (e.g., oral mucosa, rectal and intestinal mucosa, etc.) and may be administered together with other biologically active agents.

Administration can be systemic or local. In addition, it may be desirable to introduce the pharmaceutical compositions and peptide mimetics of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

Preferably, pharmaceutical compositions and peptide mimetics of the invention may be administered locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a peptide mimetic, of the invention, care must be taken to use materials to which the protein does not absorb. In another embodiment, the compound or composition can be delivered in a vesicle, in particular a liposome. Preferably, the composition can be delivered in a controlled release system. Preferably the composition can be delivered in a sustained release system. In one embodiment, a pump may be used. In another embodiment, polymeric materials can be used. Preferably, a controlled release system can be placed in proximity of the therapeutic target, thus requiring only a fraction of the systemic dose.

### Other Uses

The activities of a variety of β-TrCP substrates, including the peptide mimetics of DKK3b in accordance with the invention can also be used as biomarkers or companion diagnostics for Dkk3b treatments, (e.g., blood cells could be collected from patients pre- and post-AC1 treatment). TNF or phorbol ester (PBA), or lipopolysaccharide (LPS) could be used to stimulate NF-kB activity in the collected blood cells. The pre- versus post-treatment ratio NF-kB dependent cell activity would indicate AC1 activity.

### Production of Peptide Mimetics

The peptide mimetics can be produced recombinantly or by chemical methods for peptide synthesis.

The invention provides nucleic acid sequences encoding a peptide mimetic of the invention for recombinant production. Preferably the nucleic acid sequence is codon optimized for expression in the host cell type selected for expression. The nucleic acid sequence encoding a peptide mimetic of the invention is inserted into an appropriate expression vehicle, that is, a vector which contains the necessary elements for the transcription and translation of the inserted coding sequence, or in the case of an RNA viral vector, the necessary elements for replication and translation. The expression vehicle is then transfected into a suitable host cell which will express the peptide mimetic.

Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

Optionally, the vector may contain a “tag”-encoding sequence, that is, an oligonucleotide molecule located at the 5′ or 3′ end of the coding sequence, the oligonucleotide sequence encoding polyHis (such as hexaHis), or another “tag” for which commercially available antibodies exist, such as FLAG®, HA (hemaglutinin from influenza virus), or myc. The tag is typically fused to the antibody protein upon expression and can serve as a means for affinity purification of the antibody from the host cell. Affinity purification can be accomplished, for example, by column chromatography using antibodies against the tag as an affinity matrix. Optionally, the tag can subsequently be removed from the purified antibody polypeptide by various means such as using certain peptidases for cleavage.

The transformed host cell, when cultured under appropriate conditions, synthesizes a peptide mimetic that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Preferably, the host cells are selected from the group consisting of mammalian cells, bacterial cells, plant, microbial, algal and fungal cells. In some embodiments, the cells are mammalian cells, such as, but not limited to, human, mouse, rat, goat, horse, rabbit, hamster or cow cells. Preferably, the cells may be from an established cell line, including, but not limited to, HeLa, NSO, SP2/0, KEK 293T, Vero, Caco, Caco-2, MDCK, COS-1, COS-7, K562, Jurkat, CHO-K1, DG44, CHOK1SV, CHO-S, Huvec, CV-1, Huh-7, NIH3T3, HEK293, 293, A549, HepG2, MCF-7, U-20S, Per.C6, SF9, SF21 or Chinese Hamster Ovary (CHO) cells.

Preferably, the nucleic acid encoding the peptide mimetic further encodes an exogenous signal sequence effective to deliver the expressed peptide mimetic to the secretory pathway of the cell thereby allowing recovery and purification of the peptide mimetic from the cell culture medium. If the peptide mimetic is expressed without a secretory signal peptide, it may be necessary to recover the peptide mimetic from host cell lysates.

Preferably the secretory recognition peptide (SRP) is any well-known sequence motif that targets proteins for translocation across the endoplasmic reticulum (ER) membrane. SRPs are generally derived from secreted proteins and may be further modified. Preferred SPs the SP from Azurocidin, the SP from the PTEN (Phosphatase and Tensin Homologue on chromosome Ten), the SP from heparin binding protein (HPB).

Preferably the peptide mimetic is further purified from and/or concentrated in the culture media. In some embodiments, peptide mimetic is purified and/or concentrated using a suitable method. Suitable methods include, reverse phase chromatography, high performance liquid chromatography, ion exchange chromatography, size exclusion chromatography, affinity chromatography, gel electrophoresis, and the like. The actual conditions used to purify a particular peptide mimetic will depend, in part, on synthesis strategy and on factors such as net charge, hydrophobicity, hydrophilicity, and the like, and will be apparent to those of ordinary skill in the art.

An example of a peptide mimetic of the invention that includes an SRP and a poly-His purification tag has the following amino acid sequence:

(SEQ ID NO: 50)
MTRLTVLALLAGLLASSRAGSGRGHHHHHHVGTGSNSPGMDAEDLLLKLN
LAATVGTAPPKGKNLGQAYPASSDKESEVGRYSHSPHQGSSACMVARRRR
RRAHRDGMACPSTRSNNGIAIPVPTAALLIILGGDDI.

As is understood in the art, the SRP and purification tags are cleaved prior to using the peptide mimetic in the pharmaceutical compositions and methods of the invention. Exemplary, non-limiting methods for chemically synthesizing peptides (referred to herein as “peptide synthesis”) include those described by Stuart and Young in “Solid Phase Peptide Synthesis,” Second Edition, Pierce Chemical Company (1984), “Solid Phase Peptide Synthesis,” Methods Enzymol. 289, Academic Press, Inc, New York (1997), Proteins; Structures and molecular properties, second edition (1993) W. H. Freeman and Company, Merrifield, B. “Solid phase synthesis” Science (1986)232, 241-247; and Sheppard, R. C., “Modern Methods of solid phase peptide synthesis” Science Tools (1986) 33, 9-16; each reference incorporated herein by reference in its entirety. Such methods include using solution and solid phase chemical peptide synthesis. For example, the peptides and peptide mimetics described herein may be synthesized using solid phase strategies on an automated multiple peptide synthesizer (Abimed AMS 422) using 9-fluorenylmethyloxycarbonyl (Fmoc) chemistry. The purities of various synthetic preparations can be assessed by, for example, high-performance liquid chromatography analysis and mass spectroscopy. Chemical synthesis approaches may be advantageous over cellular expression systems (e.g., yeast or bacteria protein expression systems), as they may preclude the need for extensive recombinant protein purification steps (e.g., required for clinical use). In contrast, longer synthetic polypeptides may be more complicated and/or costly to produce via chemical synthesis approaches and such polypeptides may be more advantageously produced using cellular expression systems. In some embodiments, the peptides of the present description are chemically synthesized (e.g., solid- or liquid phase peptide synthesis). In some embodiments, the peptides, e.g., the peptide mimetics and the N-terminal peptides of the present description may lack an N-terminal methionine residue, as discussed in detail above.

### Kits

The invention provides a variety of kits for conveniently and/or effectively carrying out methods of the present invention. Typically, kits will comprise sufficient amounts and/or numbers of components to allow a user to perform multiple treatments of a subj ect(s) and/or to perform multiple experiments and packaging and instructions.

In some embodiments, kits would provide split doses or instructions for the administration of split dosages of the peptide mimetic of the kit.

### Equivalents and Scope

Those skilled in the art will recognize or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein.

Any particular embodiment of the compositions of the invention; any method of production; any method of use can be excluded from any one or more claims, for any reason, whether or not related to the existence of prior art.

### EXAMPLES

### Example 1: Comparison of β-Catenin Silencing Activity between the Peptide Mimetic, AC1 and cpDKK3b

The amino acid sequence of the peptide mimetic, AC1, was synthesized using a double stranded cDNA g-block comprised of an (i) initiator methionine followed by (ii) the secretion domain of human azurocidin, (iii) a 4 amino acid spacer, (iv) six histidine residues, (v) a 9 random amino acid spacer and (vi) SEQ ID NO: 1:

(SEQ ID NO: 50)
MTRLTVLALLAGLLASSRAGSGRGHHHHHHVGTGSNSPGMDAEDLLLKLN
LAATVGTAPPKGKNLGQAYPASSDKESEVGRYSHSPHQGSSACMVARRRR

RRAHRDGMACPSTRSNNGIAIPVPTAALLIILGGDDI;

wherein (i)-(v) are italicized.

This cDNA was cloned into the pcDNA3.4 expression vector. Expression of the secreted AC1 was done using ExpiCHO-S cells. The secreted AC1 was purified by IMAC chromatography using Ni-NTA resin.

AC1 (SEQ ID NO:1) and the fusion peptide, cpDKK3b (SEQ ID NO: 2) were evaluated for β-Catenin signaling activity in a TOPFLASH assay.

TOPLFASH reporter cells were generated by inserting the Lef/Tcf-Luc2CP reporter construct into the genome of HEK293 cells using a replication deficient lentivirus. β-catenin signaling was induced by adding 30 mM LiCl and increasing concentrations of AC1 or cpDKK3b was added to the culture medium. After 16 h at 37° C., luciferase activity was measured using a commercial luciferase assay. Treatment experiments were performed in quadruplicate, and each experiment was repeated at least three times.

Results are shown in FIG. 2. AC1 is 10,000-fold more potent that cpDKK3b with regard to β-Catenin Silencing activity.

Without being limited to any theory, it is believed that the design of the peptide mimetics of the invention including AC1 allow the peptide mimetics disclosed herein to fold into a configuration that is closely related to the folding of the native DKK3b protein (SEQ ID NO: 47). cpDKK3b is a denatured molecule which dramatically increases the protein load required for therapeutic efficacy.

### Example 2: Comparison of Effects of AC1 and cpDKK3b on Cancer Cell Survival

Quadruplicate wells of a poly-lysine coated 96 well microtiter dish were seeded with 10,000 cells of Ovarian Cancer (OVCAR3) or Colorectal Cancer (Colo205) cells and grown for 24 h at 37C. Increasing concentrations of AC1 or cpDKK3b was added to the culture medium and the cells were grown for an additional 16 h at 37° C. Treatment medium was gently aspirated, wells washed 3 times with cold phosphate buffered saline, and cell DNA was labeled with 5μM DRAQS. DNA in individual wells were imaged using the 700 nm channel of an LiCOR Odyssey Clx scanner. Data were processed using Image Studio Ver 5.2 software (LiCOR). Data are reported as means of quadruplicate wells. The results are shown in FIG. 3. AC1 is 10,000-fold more potent that cpDKK3b with regard to inhibition of cancer cell growth.

### Example 3: Bioavailability of Peptide Mimetic of SEQ ID NO: 1

A in vivo bioreporter gene, a short-lived beta catenin-driven firefly luciferase cDNA, was introduced into Human Ovarian cancer (OVCAR3) cells using lentivirus infection. Bioreporter expressing, puromycin resistant OVCAR3 cells (OVR3R cells) were selected with antibiotic and expanded. 5×10⁶ OVR3R cells were suspended in 100 μl of 50% Matrigel and injected (subcutaneous) in the flank of immune comprised nude mice. Tumors were allowed to grow to 100 mm³ (range 85-105 mm³) prior to study.

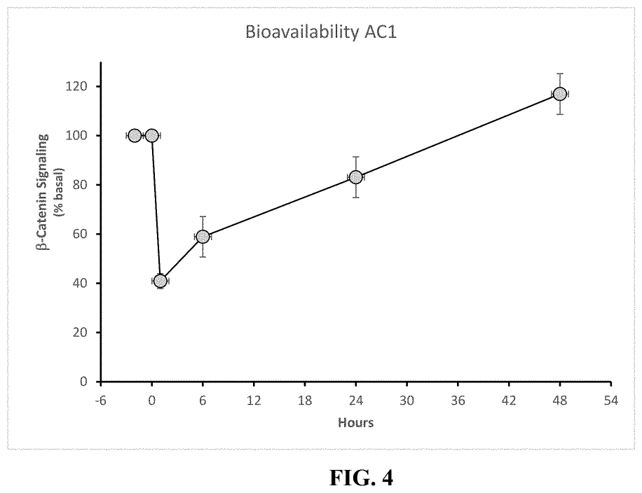

Prior to injection of AC1, tumor localized beta-catenin signaling was imaged using IVIS Spectrum series bioluminescence optical imaging system and injected VivoGlo Luciferin. At the start of the study AC1 (0.5 μg) was injected IP in 200 μl of PBS and tumor bioluminescence (BIL) was measured at 1, 6, 24 and 48 hours after injection. BIL-total flux (photons/s)—was measured for each OVR3R tumor over time and the BIL at each time normalized to the basal level. Data are reported as the means±se, n=6. The results are shown in FIG. 4.

### Example 4: Peptide Mimetic AC2 Provides Time-Dependent Inhibition of Beta-Catenin Signaling In Vivo

Ovarian Cancer cells (5×10⁶ OVCAR3 cells expressing a beta catenin dependent luciferase cDNA), were implanted under the flank skin in 5 nude mice and tumors were allowed to grow to a volume of >100 mm³. Tumor beta catenin signaling was measured using in vivo imaging by IVIS Spectrum imager (PerkinElmer, model: IVIS Spectrum Preclinical In Vivo Imaging System).

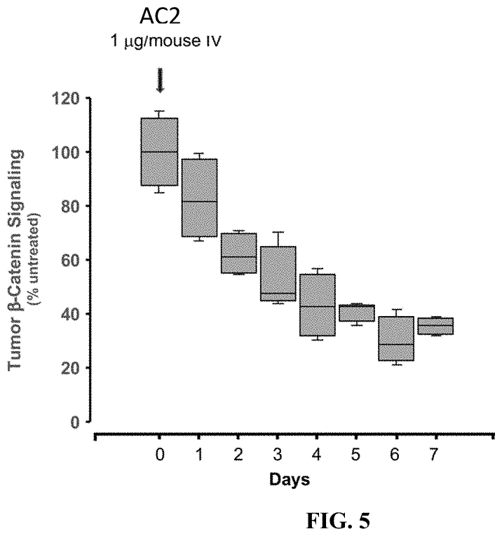

On day 0 of the study, each mouse received a single 100 microliter i.v. injection of peptide mimetic AC2 (which has the amino acid sequence of SEQ ID NO: 75) containing 1 microgram of drug in physiological saline supplemented with 100 micrograms per milliliter dextran sulfate (5,000 mw). Mice were imaged daily. The results of the study are shown in FIG. 5 which provide data from five mice. As shown in FIG. 5, the injected peptide mimetic reaches the implanted tumor and shows a time dependent decrease beta catenin signaling that plateaus after 4 days with a 60% beta catenin silencing.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

1. A peptide mimetic of human DKK3b comprising:
i. an N-terminal domain having an amino acid sequence that comprises a random coil, α-helix, or β-pleated sheet and comprising about 2 to about 3 negatively charged amino acids within the first 6 amino acids of the N-terminal domain;
ii. an N-1 domain that is a variant of the N-1 domain of human DKK1, DKK2, DKK3b, or DKK4, wherein the variant is at least about 75% identical to the N-1 domain of human DKK1, DKK2, DKK3b, or DKK4, and wherein the variant comprises a cell-penetrating peptide, or
an N-1 domain that comprises an amino acid sequence that is at least about 80% identical to SEQ ID NO: 7, 8, 45 ,46, or 69, and wherein the N-1 domain comprises a cell-penetrating peptide; and
iii. a C-terminal domain having an amino acid sequence comprising a random coil, alpha helix, or β-pleated sheet comprising about 2 to about 3 negatively charged amino acids within the last 6 amino acids of the C-terminal domain;
and wherein the peptide mimetic is an inhibitor of β-catenin nuclear translocation or a β-catenin signaling pathway.

2. (canceled)

3. The peptide of claim 1, wherein the cell penetrating domain is a peptide of about 4 to about 8 Arginine residues in length.

4. The peptide mimetic of claim 1, wherein the peptide mimetic comprises the N-1 domain that is a variant of the N-1 domain of one of human DKK1, DKK2, DKK3b, and DKK4 having the amino acid sequence of SEQ ID NO: 3, 4, 5 and 6, respectively, wherein one or more cysteine residues of the N-1 domain of human DKK1, DKK2, DKK3b, or DKK4 are substituted with a conservative amino acid.

5. The peptide mimetic of claim 4, wherein the N-1 domain comprises an amino acid sequence that is at least about 80% identical to SEQ ID NOs: 7, 8, 45, 46, or 69 and wherein the N-1 domain comprises a cell-penetrating peptide.

6. The peptide mimetic of claim 5, wherein the N-1 domain comprises an amino acid sequence that is at least about 80% identical to SEQ ID NO: 45.

7. The peptide mimetic of claim 1, comprising an amino acid linker between the N-terminal domain and the N-1 domain and/or between the N-1 domain and the C-terminal domain.

8. (canceled)

9. (canceled)

10. The peptide mimetic of claim 1, wherein the N-terminal domain comprises negatively charged amino acids at amino acid positions 2, 4 and 5.

11. The peptide mimetic of claim 1, wherein the N-terminal domain comprises an amino acid sequence that is at least about 80% identical to SEQ ID NO: 59.

12. The peptide mimetic of claim 1, wherein the N-terminal domain comprises the amino acid sequence of SEQ ID NO: 59.

13. The peptide mimetic of claim 1, wherein the N-terminal domain comprises the amino acid sequence of Φ₁DAEDLLLKLNLAATVGTAPP (SEQ ID NO: 62), wherein Φ₁ is threonine, serine, or a nonpolar amino acid other than proline and methionine.

14. The peptide of claim 13, wherein the N-terminal domain comprises an amino acid sequence selected from the group consisting of ADAEDLLLKLNLAATVGTAPP (SEQ ID NO: 63) and IDAEDLLLKLNLAATVGTAPP (SEQ ID NO: 64).

15. The peptide mimetic of claim 1, wherein the C-terminal domain comprises two, consecutive, negatively-charged amino acids within the last 6 amino acids of the C-terminal domain.

16. The peptide mimetic of claim 15, wherein the negatively-charged amino acids are positioned just prior to the last amino acid of the C-terminal domain.

17. The peptide mimetic of claim 1, wherein the C-terminal domain comprises an amino acid sequence that is at least about 80% identical to SEQ ID NO: 49 or an amino acid sequence that is at least about 80% identical to SEQ ID NO: 60.

18. (canceled)

19. The peptide mimetic of claim 17, wherein the C-terminal domain comprises the amino acid sequence of SEQ ID NO: 60.

20. The peptide mimetic of claim 1, comprising an amino acid sequence that is at least about 80% identical to the amino acid sequence of SEQ ID NO: 1.

21. The peptide mimetic of claim 20, comprising the amino acid of SEQ ID NO: 1.

22. The peptide mimetic of claim 1, comprising an amino acid sequence that is at least about 80% identical to the amino acid sequence of:
(SEQ ID NO: 66)
ϕ₂DAEDLLLKLNLAATVGTAPPKGKNLGQAYPASSDKESEVGRYSHSPHQ
GSSACMVARRRRRRAHRDGMACPSTRSNNGIAIPVPTAALLIILGGDDI,
or
(SEQ ID NO: 70)
ϕ₂DAEDLLLKLNLAATVGTAPPKGKNLGQAYPASSDKESEVGRYSHSPHQ
GSSAωMVARRRRRRAHRDGMAωPSTRSNNGIAIPVPTAALLIILGGDDI;
wherein Φ₂ is threonine, serine, or a nonpolar amino acid other than proline and methionine; and wherein each ω is independently alanine or serine.

23. The peptide mimetic of claim 22, wherein the peptide mimetic comprises an amino acid sequence selected from the group consisting of:
(SEQ ID NO: 67)
ADAEDLLLKLNLAATVGTAPPKGKNLGQAYPASSDKESEVGRYSHSPHQG
SSACMVARRRRRRAHRDGMACPSTRSNNGIAIPVPTAALLIILGGDDI;
(SEQ ID NO: 68)
IDAEDLLLKLNLAATVGTAPPKGKNLGQAYPASSDKESEVGRYSHSPHQG
SSACMVARRRRRRAHRDGMACPSTRSNNGIAIPVPTAALLIILGGDDI;
(SEQ ID NO: 75)
IDAEDLLLKLNLAATVGTAPPKGKNLGQAYPASSDKESEVGRYSHSPHQG
SSASMVARRRRRRAHRDGMAAPSTRSNNGIAIPVPTAALLIILGGDDI;
(SEQ ID NO: 76)
IDAEDLLLKLNLAATVGTAPPKGKNLGQAYPASSDKESEVGRYSHSPHQG
SSAAMVARRRRRRAHRDGMASPSTRSNNGIAIPVPTAALLIILGGDDI;
(SEQ ID NO: 77)
ADAEDLLLKLNLAATVGTAPPKGKNLGQAYPASSDKESEVGRYSHSPHQG
SSASMVARRRRRRAHRDGMAAPSTRSNNGIAIPVPTAALLIILGGDDI;
and
(SEQ ID NO: 78)
ADAEDLLLKLNLAATVGTAPPKGKNLGQAYPASSDKESEVGRYSHSPHQG
SSAAMVARRRRRRAHRDGMASPSTRSNNGIAIPVPTAALLIILGGDDI.

24-45. (canceled)

46. A pharmaceutical composition comprising the peptide mimetic of claim 1 and a pharmaceutically acceptable carrier.

47. A method of inhibiting nuclear translocation of β-catenin in a patient in need thereof, the method comprising administering an effective amount of a peptide mimetic of claim 1 to the patient.

48-51. (canceled)
